(19) United States
(12) Patent Application Publication
(10) Pub. No.: US 2014/0128712 A1
(43) Pub. Date: May 8, 2014

(54) SYSTEM FOR ELECTROPHYSIOLOGY THAT INCLUDES SOFTWARE MODULE AND BODY-WORN MONITOR

(71) Applicant: Perminova Inc., La Jolla, CA (US)

(72) Inventors: Matt Banet, Kihei, HI (US); Greg Feld, Rancho Santa Fe, CA (US); Marshal Dhillon, San Diego, CA (US); Drew Terry, San Diego, CA (US)

(73) Assignee: Perminova Inc., La Jolla, CA (US)

(21) Appl. No.: 14/073,597

(22) Filed: Nov. 6, 2013

Related U.S. Application Data
(60) Provisional application No. 61/723,168, filed on Nov. 6, 2012.

(51) Int. Cl. A61B 5/02; A61B 5/0408

(52) U.S. Cl. CPC A61B 5/02; A61B 5/0408
USPC 600/391; 600/513

(57) ABSTRACT

The invention also provides an integrated system that combines an ablation system used in the electrophysiology (EP) lab with a novel, body-worn monitor and data-management software system. The body-worn monitor differs from conventional monitors in that it measures stroke volume (SV) and cardiac output (CO) in addition to heart rate (HR) and ECG waveforms. The combined system collects numerical and waveform data from patients before, during, and after an EP procedure, thereby providing a robust data set that can be used for a variety of analytics and reporting purposes. The body-worn monitor can be applied to the patient immediately after the EP procedure, e.g. while they are recovering in a hospital. Once applied, the body-worn monitor measures data in real-time, and transmits them to both an EMR and a software application running on a mobile device, such as a smartphone, tablet, or personal digital assistant.

### CROSS REFERENCES TO RELATED APPLICATIONS

This application claims the benefit of U.S. Provisional Application No. 61/723,168, filed Nov. 6, 2012, which is hereby incorporated in its entirety including all tables, figures, and claims.

### BACKGROUND OF THE INVENTION

The following discussion of the background of the invention is merely provided to aid the reader in understanding the invention and is not admitted to describe or constitute prior art to the present invention.

The present invention relates to systems for processing data from patients undergoing cardiovascular procedures, e.g. electrophysiology (EP) procedures.

Patients with abnormal cardiac rhythms can be treated with EP, or receive an implanted device (ID), such as a pacemaker or implantable cardioverter-defibrillator. These therapies and devices are effective in restoring the patient's cardiac rhythm to a normal level, and are typically characterized by a collection of data-generating devices that are used before, during, and after procedures for EP or the ID.

Prior to such a procedure, physicians often prescribe electrocardiography (ECG) monitors that measure time-dependent waveforms, from which heart rate (HR) and information related to arrhythmias and other cardiac properties are extracted. These systems can characterize ambulatory patients over short periods (e.g. 24-48 hours) using ‘holter’ monitors, or over longer periods (e.g. 1-3 weeks) using cardiac event monitors. Conventional holter or event monitors typically include a collection of chest-worn ECG electrodes (typically 3 or 5), an ECG circuit that collects analog signals from the ECG electrodes and converts these into multi-lead ECG waveforms, and a computer processing unit that analyzes the ECG waveforms to determine cardiac information. Typically the patient wears the entire system on their body. Some modern ECG-monitoring systems include wireless capabilities that transmit ECG waveforms and other numerical data through a cellular interface to an Internet-based system, where they are further analyzed to generate, for example, reports describing the patient's cardiac rhythm. In less sophisticated systems, the ECG-monitoring system is worn by the patient, and then returned to a company that downloads all relevant information into a computer, which then analyzes it to generate the report. The report, for example, may be imported into the patient's electronic medical record (EMR). The EMR avails the report to cardiologists or other clinicians, who then use it to help characterize the patient.

To monitor non-ambulatory, hospitalized patients, conventional vital sign monitors include ECG monitoring systems that characterize a patient's cardiac response in a similar way to holter or event monitors. Such monitors typically measure multi-lead ECG waveforms that are processed by embedded software within the monitor to generate ECG waveforms and determine HR and a wide range of other cardiac properties.

During a conventional EP procedure, software systems can collect physiological information from the patient (e.g. vital signs and ECG waveforms), which is then used to help guide the procedure. These data are also stored in the patient's EMR, where they can be used for future analysis by cardiologists and other clinicians. ECG systems used during EP procedures typically measure 12 leads of ECG waveforms, which a cardiologist then interprets to elucidate, diagnose, and ultimately treat the electrical activities of the patient's heart. Additionally, during EP, an invasive catheter records spontaneous activity of the heart, as well as cardiac responses to programmed electrical stimulation (PES). In addition to these diagnostic and prognostic procedures, an EP cardiologist uses therapeutic methods, such as radio frequency ablation of pre-determined portions of the heart, to adjust the patient's cardiac rhythm to a relatively stable state. ECG-monitoring devices used in the EP procedure measure the response of the injured or cardiomyopathic myocardium to PES or specific pharmacological regimens in order to assess the likelihood that the regimen will successfully prevent potentially fatal sustained ventricular tachycardia (VT) or ventricular fibrillation (VF) in the future. Sometimes a series of drug trials are conducted before and/or after an EP procedure to enable the cardiologist to select a regimen for long-term treatment that best prevents or slows the development of VT or VF following PES. Other therapeutic modalities employed in this field include antiarrhythmic drug therapy and IDs. Such studies may also be conducted in the presence of a newly deployed ID.

Many conventional EMRs are large software systems hosted on computer servers within a hospital or medical clinic. Some EMRs reside in ‘the cloud’, meaning they are hosted on remote, Internet-connected computer servers (located, e.g., in a third-party data center), which then render a graphical user interface (GUI) to hospital clinicians with a conventional web browser. In most instances, hospital administrators and clinicians use either the EMR or a secondary software system to perform ancillary functions related to the EP procedure, such as scheduling, billing, and patient follow-up.

Stroke volume (SV) is the mathematical difference between left ventricular end diastolic volume (EDV) and end systolic volume (ESV), and represents the volume of blood ejected by the left ventricle with each heartbeat; a typical value is about 80 mL. Cardiac output (CO) is the average, time-dependent volume of blood ejected from the left ventricle into the aorta and, informally, indicates how efficiently a patient's heart pumps blood through their arterial tree; a typical value is about 5 L/min. CO is the product of HR and SV, i.e.:

CO=SV×HR (1)

Measuring CO and SV in a continuous, non-invasive manner with high clinical accuracy has often been considered a ‘holy grail’ of medical-device monitoring. Most existing techniques in this field require in-dwelling catheters, which in turn can harm the patient, are inherently inaccurate in the critically ill, and require a specially trained operator. For example, current ‘gold standards’ for this measurement are thermodilution cardiac output (TDCO) and the Fick Oxygen Principal (Fick). However both TDCO and Fick are highly invasive techniques that can cause infection and other complications, even in carefully controlled hospital environments. In TDCO, a pulmonary artery catheter (PAC), also known as a Swan-Ganz catheter, is typically inserted into the right portion of the patient's heart. Procedurally a bolus (typically 10 ml) of glucose or saline that is cooled to a known temperature is injected through the PAC. A temperature-measuring device within the PAC, located a known distance away (typically 6-10 cm) from where fluid is injected, measures the progressively increasing temperature of the diluted blood. CO is then estimated from a measured time-temperature curve, called the ‘thermodilution curve’. The larger the area under this curve, the lower the cardiac output. Likewise, the smaller the area under the curve implies a shorter transit time for the cold bolus to dissipate, hence a higher CO.

Fick involves calculating oxygen consumed and disseminated throughout the patient's blood over a given time period. An algorithm associated with the technique incorporates consumption of oxygen as measured with a spirometer with the difference in oxygen content of centralized blood measured from a PAC and oxygen content of peripheral arterial blood measured from an in-dwelling cannula.

Both TD and Fick typically measure CO with accuracies between about 0.5-1.0 l/min, or about +/−20% in the critically ill.

Several non-invasive techniques for measuring CO and SV have been developed with the hope of curing the deficiencies of Fick and TD. For example, Doppler-based ultrasonic echo (Doppler/ultrasound) measures blood velocity using the well-known Doppler shift, and has shown reasonable accuracy compared to more invasive methods. But both two and three-dimensional versions of this technique require a specially trained human operator, and are thus, with the exception of the esophageal Doppler technique, impractical for continuous measurements. CO and SV can also be measured with techniques that rely on electrodes placed on the patient's torso that inject and then collect a low-amperage, high-frequency modulated electrical current. These techniques, based on electrical bioimpedance and called ‘impedance cardiography’ (ICG), ‘electrical cardiometry velocimetry’ (ECV), and ‘bioreactance’ (BR), measure a time-dependent electrical waveform that is modulated by the flow of blood through the patient's thorax. Blood is a good electrical conductor, and when pumped by the heart can further modulate the current injected by these techniques in a manner sensitive to the patient's CO. During a measurement, ICG, ECV, and BR each extract properties called left ventricular ejection time (LVET) and pre-injection period (PEP) from time-dependent ICG and ECG waveforms. A processer then analyzes the waveform with an empirical mathematical equation, shown below in Eq. 2, to estimate SV. CO is then determined from the product of SV and HR, as described above in Eq. 1.

ICG, ECV, and BR all represent a continuous, non-invasive alternative for measuring CO/SV, and in theory can be conducted with an inexpensive system and no specially trained operator. But the medical community has not embraced such methods, despite the fact that clinical studies have shown them to be effective with some patient populations. In 1992, for example, an analysis by Fuller et al. analyzed data from 75 published studies describing the correlation between ICG and TD/Fick (Fuller et al., The validity of cardiac output measurement by thoracic impedance: a meta-analysis; Clinical Investigative Medicine; 15: 103-112 (1992)). The study concluded using a meta analysis wherein, in 28 of these studies, ICG displayed a correlation of between r=0.80-0.83 against TDCO, dye dilution and Fick CO. Patients classified as critically ill, e.g. those suffering from acute myocardial infarction, sepsis, and excessive lung fluids, yielded worse results. Further impeding commercial acceptance of these techniques is the tendency of ICG monitors to be relatively bulky and similar in both size and complexity to conventional vital signs monitors. This means two large and expensive pieces of monitoring equipment may need to be located bedside in order to monitor a patient's vital signs and CO/SV. For this and other reasons, impedance-based measurements of CO have not achieved widespread commercial success.

### SUMMARY OF THE INVENTION

As described above, a collection of hardware and software systems can collect and store a patient's cardiovascular information before a cardiologist conducts a procedure for EP or an ID, during the actual procedure, and after the patient leaves the hospital or medical clinic. In theory, data during each of these phases flows into the patient's EMR. But, in reality, even state-of-the-art EMRs are only able to collect and store limited amounts of data from these systems, especially when multiple, disparate systems are used to monitor the patient. Sophisticated cardiovascular parameters, such as CO and SV, are rarely measured in these settings. And typically the data are not organized or formatted in a way that allows processing large data sets measured before, during, and after an EP procedure. Analysis of such data, if it were possible, would facilitate sophisticated inter-site clinical studies with a large number of patients. This, in turn, could yield analysis and development of new therapies, devices, and treatment protocols for cardiovascular patients.

With this in mind, the present invention provides an improved, Internet-based system that seamlessly collects cardiovascular data from a patient before, during, and after a procedure for EP or an ID. For example, during an EP procedure, the system collects information describing the patient's response to PES and the ablation process, CO, SV, ECG waveforms and their various features, HR and other vital signs, HR variability, cardiac arrhythmias, patient demographics, and patient outcomes. Once these data are collected, the system stores them on an Internet-accessible computer system that can deploy a collection of user-selected and custom-developed algorithms. A data-collection/storage module, featuring database interface, stores physiological and procedural information measured from the patient. Interfacing with the database is a data-analytics module that features a collection of algorithm-based tools run by computer code (e.g. software) that can collectively analyze information measured during each of these phases from large sets of patients. The data-analytics module also includes an Internet-based GUI that renders these data and exports them for future analysis. Patients providing data for this system may be associated with a single site, or multiple, disparate sites. Analysis of the data, for example, can yield reports that characterize the efficacy of a given procedure, or help a clinician improve a cardiac EP procedure for a given patient. In this way, the present invention can facilitate ‘virtual clinical trials’ wherein sophisticated multi-center studies are quickly and efficiently performed, all without the significant financial and time investments normally required for conventional clinical trials.

The invention also provides a highly integrated system that combines an ablation system used in the EP lab with a novel, body-worn monitor and data-management software system. The body-worn monitor differs from conventional monitors in that it measures CO and SV in addition to HR and ECG waveforms. As described above, the combined system collects numerical and waveform data from patients before, during, and after an EP procedure, thereby providing a robust data set that can be used for a variety of analytics and reporting purposes. The body-worn monitor can be applied to the patient immediately after the EP procedure, e.g. while they are recovering in a hospital. Once applied, the body-worn monitor measures data in real-time, and transmits them to both an EMR and a software application running on a mobile device, such as a smartphone, tablet, or personal digital assistant. In this manner, a clinician can use the mobile device to monitor the patient as they recover in the hospital, and then transition to the home. The system collects data continuously, thus allowing the efficacy of the EP procedure to be rapidly determined.

The body-worn monitor measures CO and SV in a continuous, non-invasive manner. These parameters indicate the mechanical performance of the patient's heart, i.e. its pumping characteristics. ECG and HR indicate the heart's electrical properties. The body-worn monitor combines these measurements into a simple, easy-to-apply device that permits evaluation of the patient's complete cardiovascular performance. Because the device is both wireless and battery-powered, the patient can move about the hospital and their home while recovering from the EP procedure, and during this period can be monitored by a supervising clinician.

The data-analytics module can perform a spectrum of calculations, ranging from simple statistical analyses (e.g. the number of EP procedures performed by a clinic, or the amount of financial reimbursement received by the clinic) to complex analysis of physiological data (e.g. Boolean searches, subsequent analyses, and image processing). Such analysis can be performed with pre-determined reporting tools, or by exporting customized data fields that can be analyzed off-line using custom algorithms.

Software associated with algorithms deployed by the data-analytics module, for example, can analyze numerical vital signs or waveforms, parameters associated with the EP procedure, parameters associated with the ID, two and three-dimensional images related to the patient's cardiovascular behavior, demographic information, and billing and financial information. These data can be analyzed, for example, to estimate or predict the condition of the patient, determine the efficacy of the EP procedure as applied to the patient, evaluate an ID and its associated components (e.g. leads), evaluate financial aspects of hospital or clinic, and evaluate demographics associated with cardiovascular issues. Alternatively, these algorithms can be used for purposes more suited to scientific research, e.g. for collectively analyzing components of ECG waveforms corresponding to large groups of patients receiving a particular EP procedure to estimate the overall efficacy of the procedure. Components of the ECG waveforms analyzed in this manner include: i) a QRS complex; ii) a P-wave; iii) a T-wave; iv) a U-wave; v) a PR interval; vi) a QRS interval; vii) a QT interval; viii) a PR segment; and ix) an ST segment. The temporal or amplitude-related features of these components may vary over time, and thus the algorithmic-based tools within the system, or software associated with the algorithm-based tools, can analyze the time-dependent evolution of each of these components. In particular, algorithmic-based tools that perform numerical fitting, mathematical modeling, or pattern recognition may be deployed to determine the components and their temporal and amplitude characteristics for any given heartbeat recorded by the system.

As an example, physiological waveforms measured with the body-worn device may be numerically ‘fit’ with complex mathematical functions, such as multi-order polynomial functions or pre-determined, exemplary waveforms. These functions may then be analyzed to determine the specific components, or changes in these components, within the waveform. In related embodiments, waveforms may be analyzed with more complex mathematical models that attempt to associate features of the waveforms with specific bioelectric events associated with the patient.

Each of the above-mentioned components corresponds to a different feature of the patient's cardiac system, and thus analysis of them according to the invention may determine or predict different cardiac conditions. These conditions and their associated components include: blockage of arteries feeding the heart (each related to the PR interval); aberrant ventricular activity or cardiac rhythms with a ventricular focus (each related to the QRS interval); prolonged time to cardiac repolarization and the onset of ventricular dysrhythmias (each related to the QT interval); P-mitrale and P-pulmonale (each related to the P-wave); hyperkalemia, myorcardial injury, myocardial ischemia, myocardial infarction, pericarditis, ventricular enlargement, bundle branch block, and subarachnoid hemorrhage (each related to the T-wave); and bradycardia, hypokalemia, cardiomyopathy, and enlargement of the left ventricle (each related to the U-wave). These are only a small subset of the cardiac conditions that may be determined or estimated through analysis of the ECG waveform according to the invention.

Algorithmic-based tools, or software associated with these tools, can also analyze relatively long traces of waveforms (spanning over seconds or minutes) measured before, during, and after the EP procedure to characterize: i) a given patient; ii) the efficacy of the EP procedure applied to that patient; iii) a given patient's need for an EP procedure; or iv) the overall efficacy of the EP procedure as applied to a group of patients. For example, analysis of relatively long traces of ECG waveforms in this manner may indicate cardiac conditions such as cardiac bradyarrhythmias, blockage of an artery feeding the heart, acute coronary syndrome, advanced age (fibrosis), inflammation (caused by, e.g., Lyme disease or Chaga's disease), congenital heart disease, ischaemia, genetic cardiac disorders, supraventricular tachycardia such as sinus tachycardia, atrial tachycardia, atrial flutter, atrial fibrillation, junctional tachycardia, AV nodal reentry tachycardia and AV reentrant tachycardia, reentrant tachycardia, Wolff-Parkinson-White (WPW) Syndrome, Lown-Ganong-Levine (LGL) Syndrome, and ventricular tachycardia. Likewise, analysis of these cardiac conditions by analyzing the ECG waveforms may indicate the efficacy of the EP procedure.

In one aspect, the invention provides a system for monitoring a patient undergoing an electrophysiology (EP) procedure. The system features: 1) a computer system comprising a database and a software environment; 2) an EP software system that generates EP information describing the patient's response to an EP procedure and transmits it to the database within the computer system; 3) a body-worn monitor configured to measure HR, SV, CO, and ECG waveforms from the patient, and transmit them to the database; and 4) an algorithm, operating in the computer system's software environment, that collectively processes the EP information, ECG waveforms, and values of HR, SV and CO to monitor the patient.

In embodiments, the algorithm collectively processes the EP information, HR, ECG waveforms, and at least one of the SV and CO values to generate an alarm corresponding to the patient. For example, the alarm is generated if the HR value exceeds a first range of values, and at least one of the SV and CO values exceeds a second range of values. Both the first and second ranges are determined directly from the EP information.

In another aspect, the invention provides a system for monitoring a patient having an ID, e.g. either a pacemaker or implantable cardioverter defibrillator. In this case, the system includes a body-worn monitor that features: 1) a first circuit for measuring analog ECG waveforms from the patient; 2) a second circuit for measuring analog thoracic bio-impedance (TBI) waveforms from the patient; and 3) a third circuit for reading information from the ID.

In embodiments, the third circuit is a ‘reader circuit’ that features a component for reading information (e.g. data relating to ECG waveforms, delivered shocks, and other proprietary information) from the implanted device. For example, in one case, the reader circuit includes a system for magnetic transduction configured to read information from the implanted device. In another, the reader circuit comprises a short-range wireless system (e.g. a short-range radio, such as Bluetooth) configured to read information over a wireless interface from the implanted device. Both the systems for magnetic transduction and short-range wireless are designed to be low-power systems that operate over very short distances.

In another aspect, the invention provides a system for characterizing a patient that features a data-processing software system that interfaces to both a treatment software system and a body-worn monitor. In this case, the data-processing software system is configured to analyze data collected during and after an invasive cardiac treatment program, e.g. an EP procedure. More specifically, the treatment software system is configured to collect data during the invasive cardiac treatment program, and the body-worn monitor is configured to measure HR and SV from the patient after the cardiac treatment program. Both these systems transmit information to the data-processing software system, which then modifies the measurement of SV using the data collected during the cardiac treatment program.

In embodiments, the treatment software system is configured to collect data describing SV during the invasive cardiac treatment program. For example, these data (e.g. secondary SV values) can be measured with a pulmonary arterial catheter. The data can be used to calibrate the measurement of SV made by the body-worn monitor. Calibration can be performed, for example, using a linear regression algorithm.

In another aspect, the invention provides a system for characterizing a patient that features a data-processing software system that interfaces to both a treatment software system and a body-worn monitor. The data-processing software system is configured to analyze data collected during and after an invasive cardiac treatment program. Here, the treatment software system collects data during the invasive cardiac treatment program, and the body-worn monitor measures ECG waveforms, HR, SV from the patient after the cardiac treatment program. Both components transmit information to the data-processing software system, which then collectively processes it during and after the invasive cardiac procedure to characterize the patient.

In embodiments, after processing the data, the data-processing software system generates a report describing the patient's cardiac performance. For example, the report can evaluate an electrical performance of the patient's heart using the ECG waveform and HR value, and the mechanical performance of the patient's heart using the SV value. In other embodiments, the report shows the time-dependent evolution of the electrical and mechanical performance of the patient's heart. The data-processing software system can also be configured to transmit the report to an electronic medical record.

In yet another aspect, the invention provides a system for evaluating an EP procedure that includes: 1) an EP software system that generates EP information describing the patient's response to an EP procedure; 2) a body-worn monitor configured to measure HR, SV, CO, and ECG waveforms from the patient; and 3) an Internet-based software system that receives and collectively analyzes EP information describing the patient's response to an EP procedure and the values of HR, SV, CO, and ECG waveforms to evaluate the EP procedure.

In embodiments, the Internet-based software system processes the EP information to determine HR and HR variability during the EP procedure, and ECG waveforms (or processed values for HR) from the body-worn monitor to determine HR and HR variability after the EP procedure. It then collectively analyses these data sets to evaluate the EP procedure. Similar ‘before and after’ analyses can be made using SV, CO, and ECG waveforms, with each being used to evaluate the efficacy of the EP procedure.

The body-worn monitor is typically worn on the patient's chest. It typically features two electrode patches, with each patch having two separate electrodes. Signals from the electrodes are multiplexed so they can be used for both ECG and TBI measurements, as is described in more detail below. The two electrodes within each patch are typically connected to a common adhesive backing. The backing typically includes a connecting member, such as a pair of metal rivets, so that the patches can snap into the body-worn monitor.

In embodiments, the body-worn monitor features two separate modules, each comprising an electronics circuit and configured to be worn in the patient's chest. The

first module houses an ECG circuit for measuring analog ECG waveforms used to calculate HR from the patient, and the second module houses a TBI circuit for measuring analog TBI waveforms used to calculate CO and SV from the patient. Typically the first and second modules connect to each other with a cable. In this configuration the body-worn module features a single analog-to-digital converter that converts the analog ECG waveforms into digital ECG waveforms, and the analog TBI waveforms into digital TBI waveforms. An internal microprocessor processes the digital ECG waveforms to determine an HR value, and the digital TBI waveforms to determine an SV value. The body-worn monitor can also include a wireless system (e.g. one using Bluetooth or WiFi chipsets) that transmits information to the computer system. Alternatively, the wireless system can transmit information to a mobile telephone, which runs a software application that transmits information to the computer system.

The invention has many advantages. In general, it combines a software system for electrophysiology with a body-worn device and mobile platform that allow a clinician to monitor a robust set of cardiovascular parameters from a recovering patient. The cardiovascular parameters feature those associated with the heart's mechanical properties (i.e. CO and SV) and electrical properties (i.e. HR and ECG). Taken collectively, these give the clinician a unique insight into the patient's condition.

Additionally, a cloud-based system, like the one described herein, that connects to the Internet from a remote server typically offers more flexibility than a system that is deployed in the same facility (e.g. a hospital or medical clinic) used to perform the EP procedure. With such a system, information from multiple, diverse patient groups can be collectively analyzed to perform sophisticated research relating to EP and other cardiovascular procedures. This facilitates ‘virtual clinical trials’, as described above, which can be conducted efficiently and inexpensively. The same system that performs the research can also generate reports and other materials using data from large groups of patients that can easily be dispersed to clinicians, thereby giving them the tools to improve their clinical practice. Moreover, Internet-based systems, i.e. systems that leverage ‘the cloud’, are inherently easier to maintain (e.g. deploy, update) compared to hosted client-server systems deployed at a collection of facilities, as new software builds and enhancements can be made on a single server, and then instantaneously deployed to multiple Internet-connected sites.

These and other advantages will be apparent from the following detailed description, and from the claims.

### BRIEF DESCRIPTION OF THE DRAWINGS

FIG. 1 shows a schematic drawing of a system according to the invention that includes an EP System for performing an EP therapy, an EP Software Module, and a body-worn Telemetry Monitor that measures CO, SV, HR, and ECG waveforms;

FIG. 2 shows a schematic drawing of the body-worn Telemetry Monitor of FIG. 1 attached to a patient's chest;

FIG. 3 shows a photograph of the body-worn Telemetry Monitor of FIG. 1, along with electrical circuitry inside each of its two modules;

FIG. 4 shows a photograph of a module of the body-worn Telemetry Monitor of FIG. 3 attaching to a custom two-part electrode;

FIG. 5 shows a drawing of a patient's chest and heart, and how the body-worn Telemetry Monitor attaches near these components to measure SV;

FIG. 6 shows a drawing of a TBI circuit for the body-worn Telemetry Monitor of FIG. 3;

FIG. 7 shows a schematic drawing of the Database of FIG. 1, featuring database tables that describe patient demographics, physiological information, and ECG waveforms collected from a patient;

FIG. 8 shows a schematic drawing of the Data Analytics module of FIG. 1, featuring an algorithm integrated with the data-collection/storage module of FIG. 2 that analyzes a patient's cardiovascular information;

FIG. 9 shows screen shots of graphical user interfaces, operating on an Apple iPhone, used for the Mobile Application of FIG. 1;

FIG. 10 shows a photograph of a graphical user interface, operating on an Android tablet, used for the Mobile Application of FIG. 1;

FIG. 11 shows an example of an operational report generated by the Data Analytics System of FIG. 1;

FIG. 12 shows a flow chart of an algorithm used to calculate SV during periods of motion;

FIG. 13 shows a mathematical derivative of a time-dependent TBI waveform;

FIG. 14 shows Bland-Altman (left) and correlation (right) graphs of SV measured with a technique similar to TBI and magnetic resonance imaging (MRI) during a clinical trial;

FIG. 15 shows a time-dependent ECG waveform measured with the ECG circuit used in the body-worn Telemetry Monitor of FIG. 3;

FIG. 16 shows a schematic drawing of a snippet taken from the time-dependent ECG waveform of FIG. 15, which graphical indications of the different components of the snippet;

FIG. 17 shows a schematic drawing of an alternate embodiment of the body-worn Telemetry Monitor of FIG. 1 that includes a ‘reader circuit’ for interrogating an ID; and

FIG. 18 shows a photograph of the body-worn Telemetry Monitor of FIG. 17, along with the reader circuit and other electrical circuitry inside each of its two modules.

### DETAILED DESCRIPTION OF THE INVENTION

The invention provides a highly integrated system that combines an ablation system used in the EP lab with a novel, body-worn monitor and data-management software system. The body-worn monitor differs from conventional monitors in that it measures CO and SV in addition to HR and ECG waveforms. In total, the combined system collects numerical and waveform data from patients before, during, and after an EP procedure, thereby providing a robust data set that can be used for a variety of analytics and reporting purposes. The body-worn monitor can be applied to the patient immediately after the EP procedure, e.g. while they are recovering in a hospital. Once applied, the body-worn monitor measures data in real-time, and transmits them to both a medical records system and a software application running on a mobile device, such as a smartphone, tablet, or personal digital assistant. In this manner, a clinician can use the mobile device to monitor the patient as they recover in the hospital, and then transition to the home. The system collects data continuously, thus allowing the efficacy of the EP procedure to be rapidly determined.

The body-worn monitor measures SV, which is the volume of blood (usually reported in units of ‘mL’) ejected from the patient's left ventricle during systole. The product of SV and HR is CO, which is the average volume of blood (usually reported in units of ‘L/min’) ejected over a predetermined period of time. These parameters indicate the mechanical performance of the patient's heart, i.e. its pumping characteristics. ECG and HR indicate the heart's electrical properties. The body-worn monitor combines these measurements into a simple, easy-to-apply device that monitors the patient's cardiovascular performance. Because the device is both wireless and battery-powered, the patient can move about the hospital and their home while recovering from the EP procedure, and during this period can be monitored by a supervising clinician.

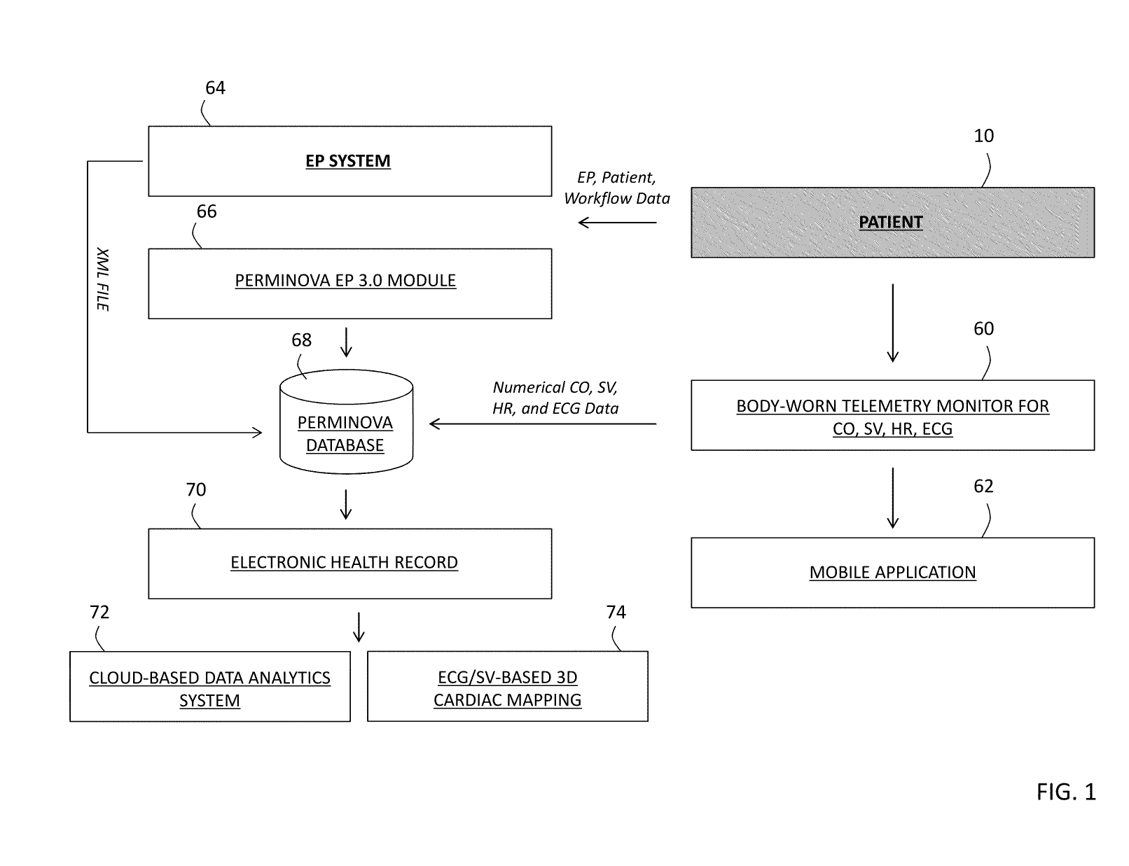

FIG. 1 provides an overview of the invention. It starts with a patient 10 monitored by an EP System 64, such as the Bard LabLink™ Data Interface, that synchronizes and integrates 3D mapping systems (e.g. the Carto® 3 System) with EP Recording Systems (e.g. the LabSystem™ PRO EP Recording System). The EP System 64 allows selection of stimulation channels from either the recording or mapping system, and merges patient demographics, 3D image snapshots and cardiovascular event data, e.g. waveforms measured with internal electrodes, refractory periods, and ablation information. During an EP procedure, the EP System 64 outputs an XML file that includes these data, encoded as either numerical values or waveforms. The XML file passes to a Database 68, where an XML parsing engine decodes it before the data elements are stored in specific fields, as described in more detail below.

An EP Module 66 also provides data for the Database 68. The EP Module 66 is preferably a system that collects information during the EP procedure, such as data describing: i) patient demographics; ii) vital signs; iii) supplies used during the EP procedure; iv) billing information; and v) clinician information. In embodiments, the EP Module is similar to that described in the co-pending patent application entitled INTERNET-BASED SYSTEM FOR COLLECTING AND ANALYZING DATA BEFORE, DURING, AND AFTER A CARDIOVASCULAR PROCEDURE (U.S. Ser. No. 61/711,096; filed Oct. 8, 2012), the contents of which are incorporated herein by reference.

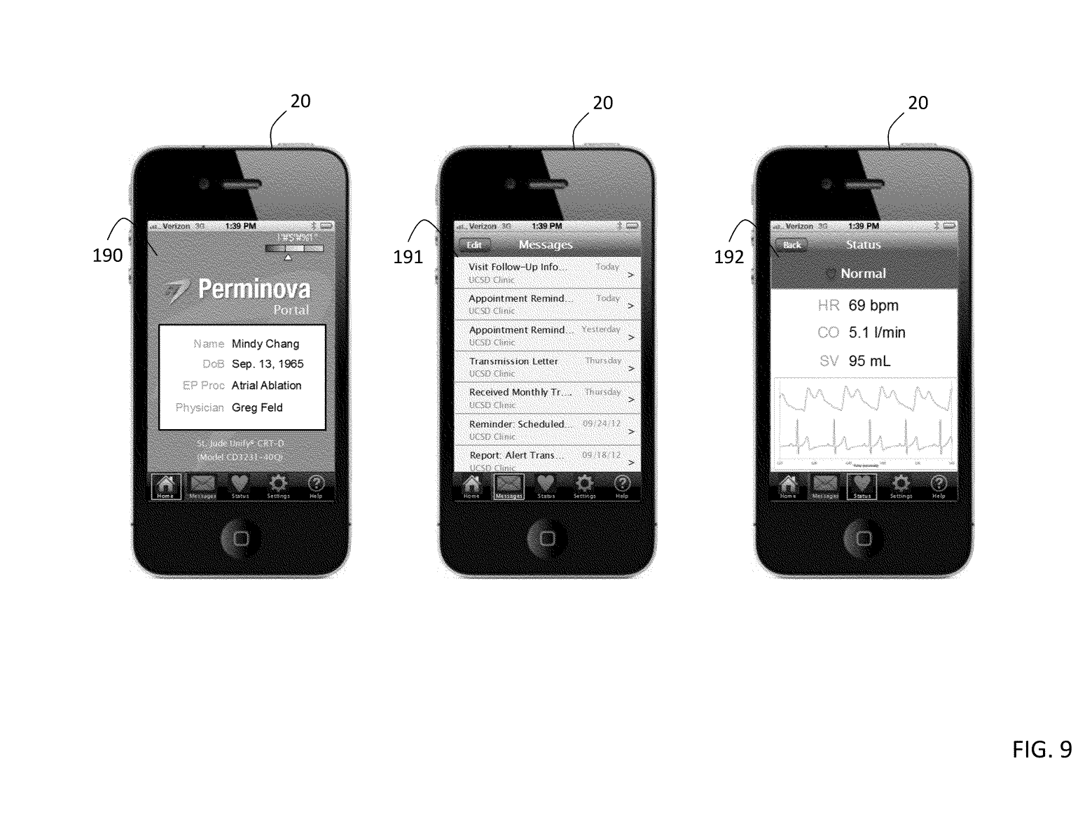

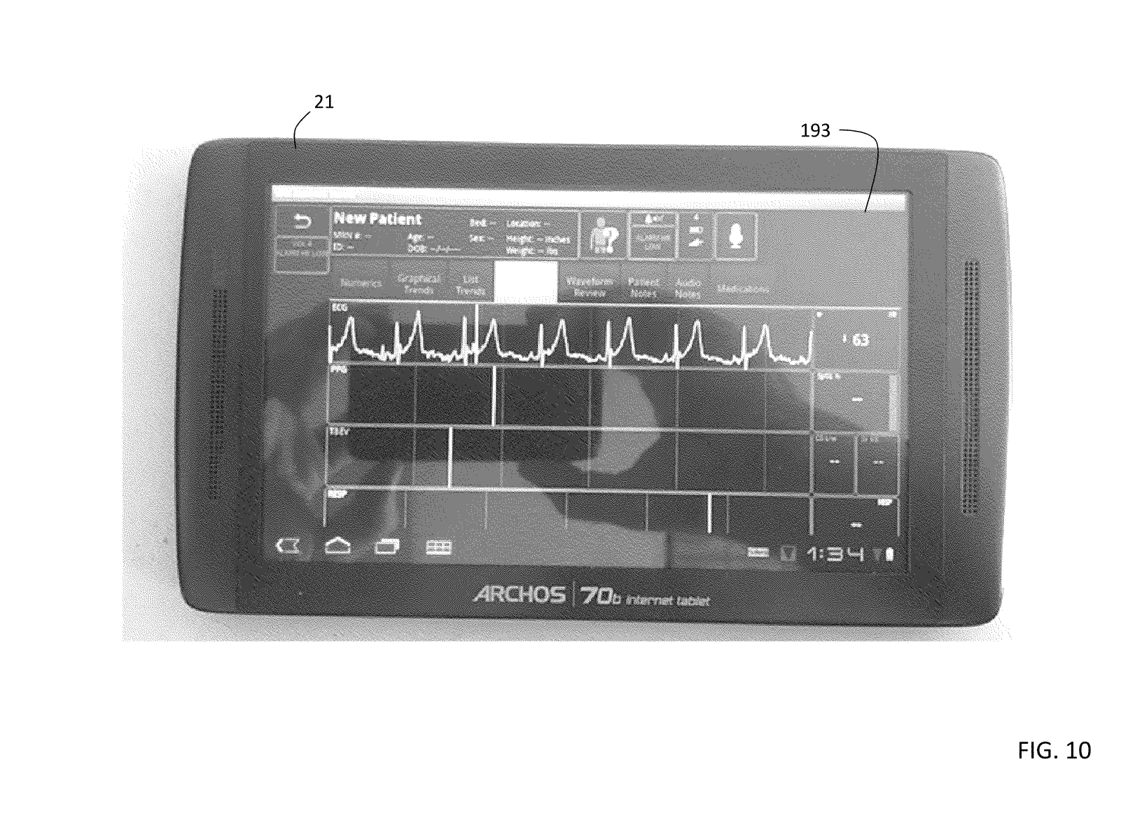

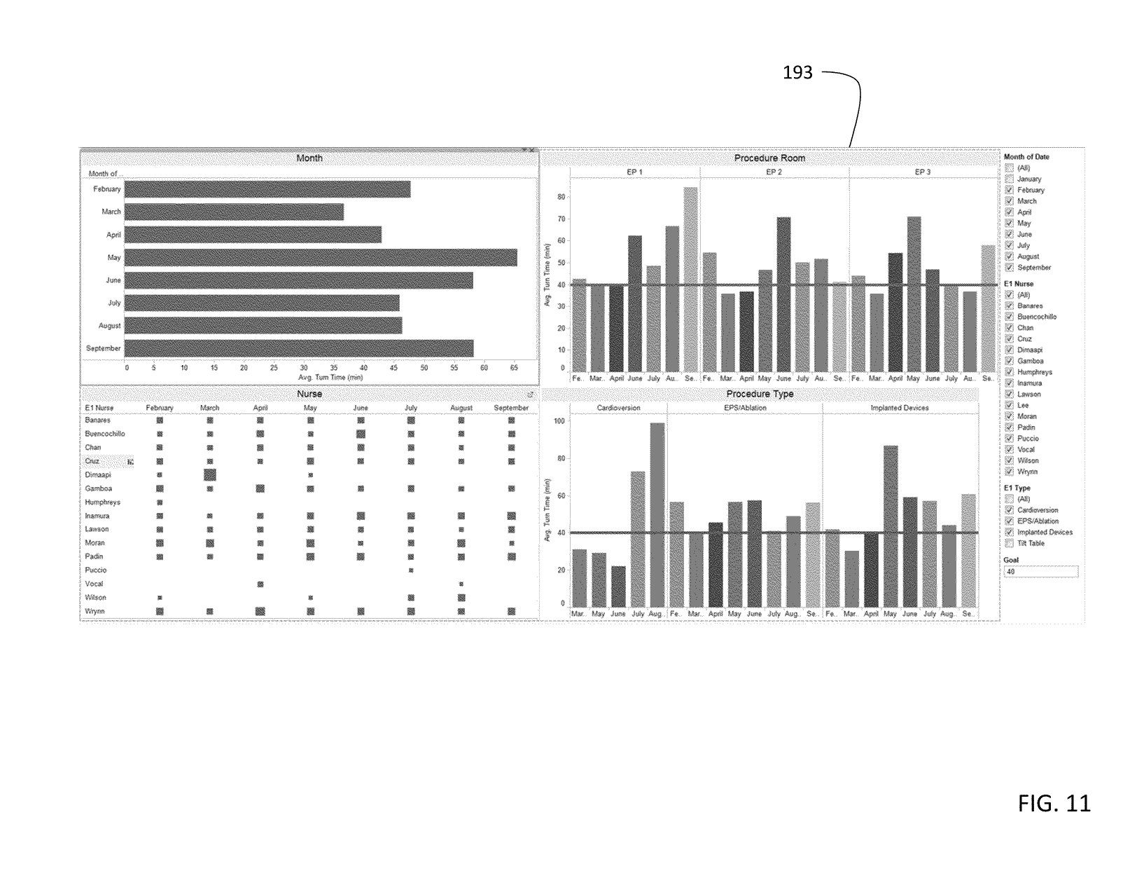

During the EP procedure, data from the EP System 64 and EP Module 66 flow from the Database 68 into the patient's Electronic Health Record 70, which is usually associated with an enterprise-level, medical-records software system deployed at the hospital, such as that provided by Epic or Cerner. Data from the Electronic Health Record 70 can be further processed by a Cloud-Based Data Analytics System 72, which is similar to that described in the above-mentioned patent application, the contents of which have been previously incorporated herein by reference. As described in this patent application, the Cloud-Based Data Analytics System 72 processes physiological, procedural, and operational data collected before, during, and after the EP procedure to generate custom reports and perform numerical studies. FIG. 11 shows an example of such an operational report. The above-referenced patent application includes several examples of how the Cloud-Based Data Analytics System 72 can process physiological data to evaluate the patient and the EP procedure overall. Additionally, a Cardiac Mapping System 74 processes CO, SV, HR, and ECG data measured by a body-worn Telemetry Monitor 60 to generate 3D images of the patient's heart. A Mobile Application 62, similar to that shown in FIGS. 9 and 10, also receives data wirelessly from the body-worn Telemetry Monitor 60, described in detail below, thereby allowing a clinician to remotely monitor the patient 10.

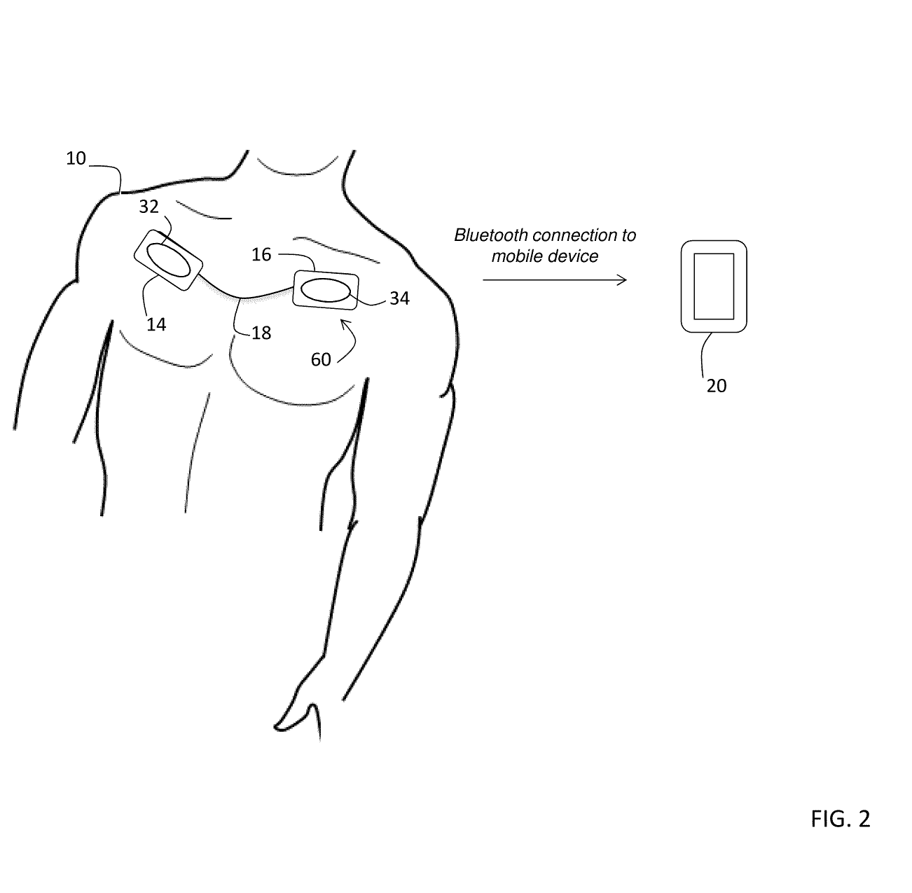

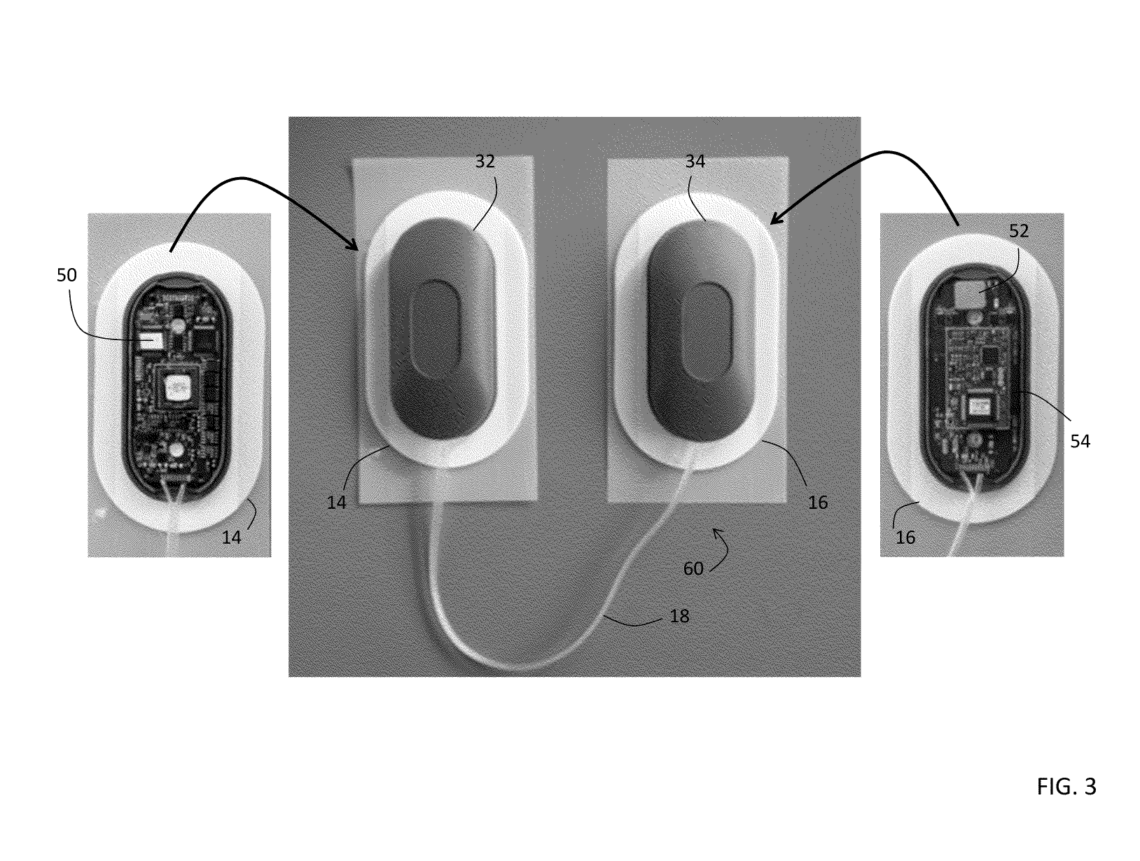

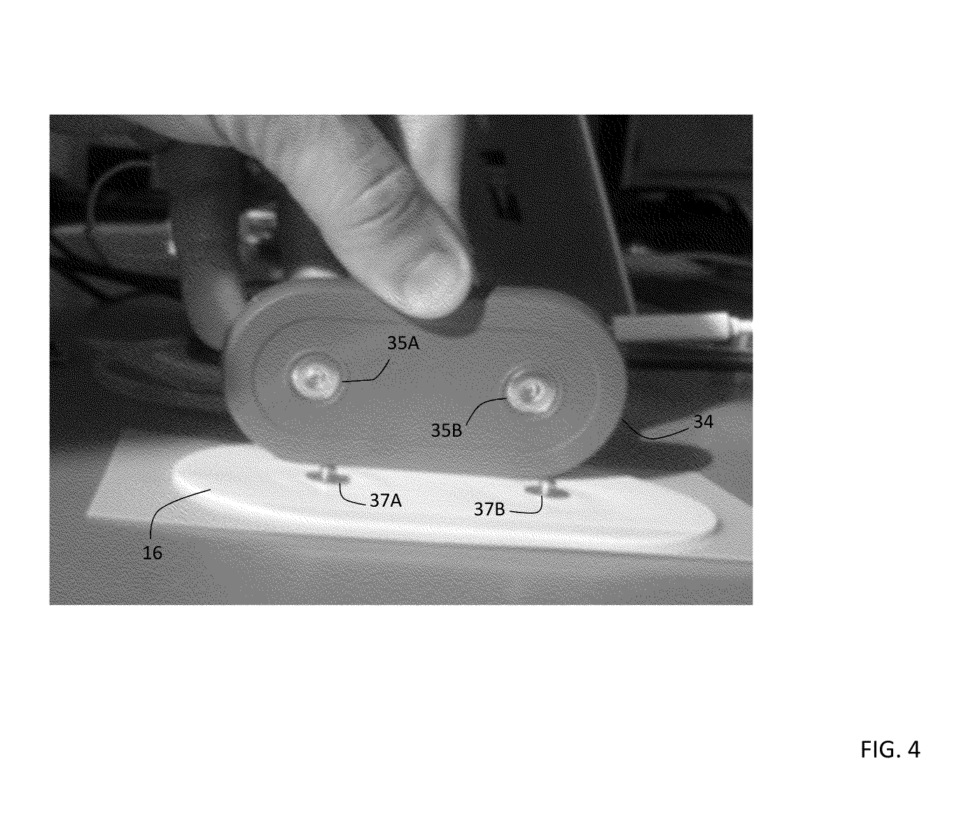

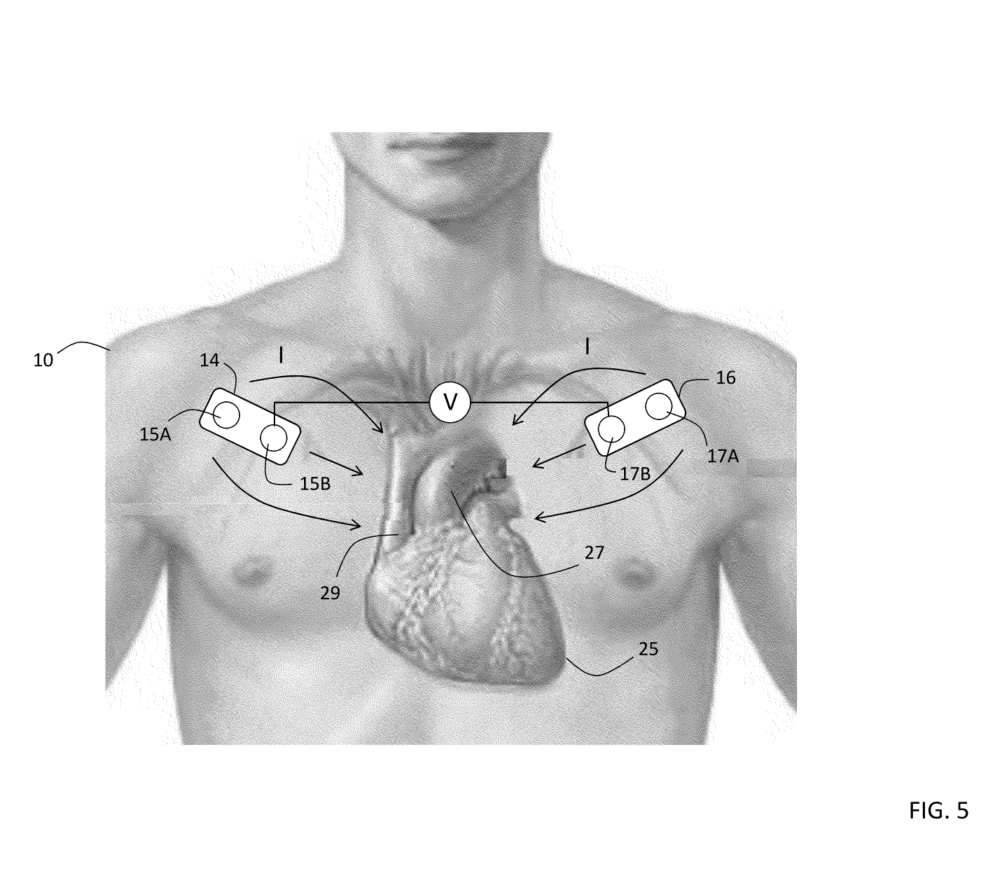

FIGS. 2, 3 show components within the Telemetry Monitor 60, and how they attach to the patient's chest to measure CO, SV, ECG, and HR. The Monitor 60 includes two separate modules 32, 34, each attached to the patient's chest with a custom, 2-part electrode 14, 16. A four-wire cable 18 connects the modules 32, 34 to supply power, ground, and transfer analog signals. More specifically, the module 32 on the patient's right-hand side includes a circuit 50 for making a TBI measurement, described in more detail below, particularly with respect to FIG. 5. The module 34 on the patient's left-hand side includes an ECG circuit 54 for measuring ECG waveforms and HR values, and a Bluetooth module 52 for wirelessly transmitting numerical and waveform data to the Mobile Application. Batteries (not shown in the figures) are included in each module 32, 34 to power the corresponding circuitry. As shown in FIG. 4, on its bottom surface, each module (module 34 is shown in the figure, and has an identical form factor to module 32) includes two snaps 35A, 35B that pop into mated rivets 37A, 37B on the top surface of the two-part electrode 16. Each rivet 37A, 37B electrically connects to a separate conductive region of the two-part electrode 16 that, in turn, attach to the patient's skin. The conductive region is composed of a standard electrode material (e.g. Ag/AgCl coating on the rivet's underside; this contacts a conductive solid gel) designed to collect bio-electric signals from the patient's chest into the TBI circuit.

FIGS. 3 and 5 indicate in more detail how the Telemetry Monitor 60 measures SV and CO from a patient. As described above, the modules 32, 34 attach to the patient's chest using the two-part electrodes 14, 16. Ideally, each module 32, 34 attaches just below the collarbone near the patient's left and right arms. During a measurement, the TBI circuit injects a high-frequency, low-amperage current (I) through outer electrodes 15A, 17A. Typically the modulation frequency is about 70 kHz, and the current is about 4 mA. The current injected by each electrode 15A, 17A is out of phase by 180°. It encounters static (i.e. time-independent) resistance from components such as bone, skin, and other tissue in the patient's chest. Additionally, blood conducts the current to some extent, and thus blood ejected from the left ventricle of the heart 25 into the aorta 27 offers a dynamic (i.e. time-dependent) resistance. The aorta 27 is the largest artery passing blood out of the heart, and thus it has a dominant impact on the dynamic resistance; other vessels, such as the superior vena cava 29, will contribute in a minimal way to the dynamic resistance.

Inner electrodes 15B, 17B measure a time-dependent voltage (V) that varies with resistance (R) encountered by the injected current (I). This relationship is based on Ohm's Law (V=I×R). During a measurement, the time-dependent voltage is measured with an analog-to-digital converter within the TBI circuit. This voltage is then processed with the well-known Sramek-Bernstein equation, or a mathematical variation thereof, to calculate SV. Historically parameters extracted from TBI signals are fed into the equation, shown below, which is based on a volumetric expansion model taken from the aortic artery:

$$SV = δ \frac{{L}^{3}}{4.25} \frac{{( d Z / d t )}_{max}}{{Z}_{0}} LVET \qquad ( 2 )$$

In Eq. 2 δ represents compensation for body mass index, Zo is the base impedance, L is estimated from the distance separating the current-injecting and voltage-measuring electrodes on the thorax, and LVET is the left ventricular ejection time, which can be determined from the TBI waveform, or from the HR using an equation called ‘Weissler's Regression’, shown below in Eq. 3, that estimates LVET from HR.

LVET=−0.0017×HR+0.413 (3)

Weissler's Regression allows LVET, to be estimated from HR determined from the ECG waveform. This equation and several mathematical derivatives are described in detail in the following reference, the contents of which are incorporated herein by reference: Bernstein, Impedance cardiography: Pulsatile blood flow and the biophysical and electrodynamic basis for the stroke volume equations; J Electr Bioimp; 1: 2-17 (2010). Both the Sramek-Bernstein Equation and an earlier derivative of this, called the Kubicek Equation, feature a ‘static component’, Z₀, and a ‘dynamic component’, ΔZ(t), which relates to LVET and a (dZ/dt)ₘₐₓ/Zₒ value, calculated from the derivative of the raw TBI signal, ΔZ(t). These equations assume that (dZ/dt)ₘₐₓ/Zₒ represents a radial velocity (with units of Ω/s) of blood due to volume expansion of the aorta.

The cable 18 connecting the two modules 32, 34 includes 4 wires. A first wire transmits a modulated current from the TBI circuit to the outer electrode 17A in the two-part electrode 16, where it is then injected into the patient's chest. The second wire connects the inner electrodes 15B, 17B in the two-part electrodes, and is used to measure the analog voltage that is ultimately used to calculate SV as described above. A third wire connects grounds between batteries included in each module 32, 34; power lines are not connected. During use, a first battery in the right-hand module 32 powers the TBI circuit, while a second battery in the left-hand module 34 powers the ECG circuit and Bluetooth module.

The inner electrodes 15B, 17B serve two purposes: 1) they measure a time-dependent voltage for the TBI measurement, as described above; and 2) they measure differential voltage signals for the ECG measurement. To accomplish this multiplexed measurement, a field effect transistor (FET) associated with the TBI circuit rapidly and periodically connects these electrodes to the TBI circuit to measure a voltage used to calculate SV, and then to the ECG circuit to measure a differential voltage that results in an ECG waveform. These connections switch back and forth with the FET at a rate of about 500 Hz, resulting in a sampling rate of 250 Hz for both the TBI and ECG measurements. Low-pass analog filters in both the TBI and ECG circuits smooth out any aberrations in the TBI and ECG waveform caused by this switching event.

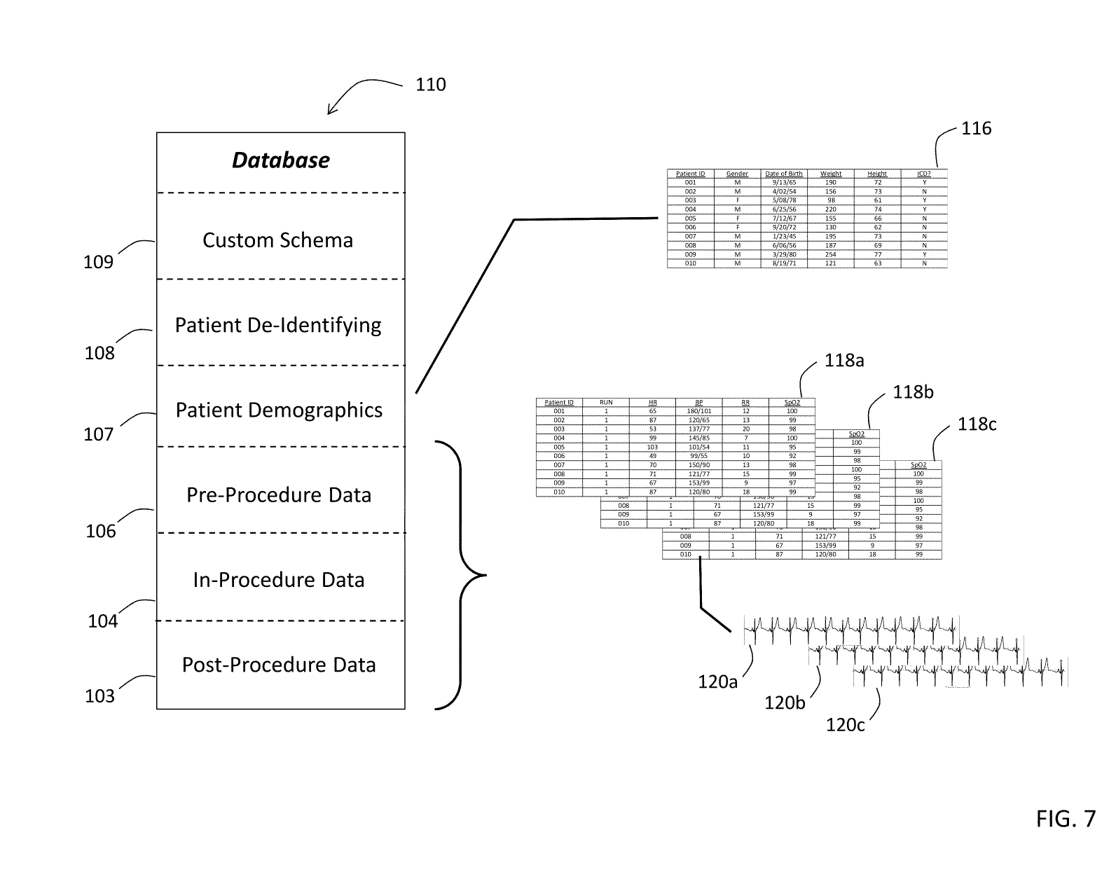

Within the right-hand module is an analog circuit 100, shown in FIG. 7, that performs the TBI measurement according to the invention. The figure shows just one embodiment of the circuit 100; similar electrical results can be achieved using a design and collection of electrical components that differ from those shown in the figure.

The circuit 100 features a first electrode 15A that injects a high-frequency, low-amperage current (I₁) into the patient's brachium. This serves as the current source. Typically a current pump 102 provides the modulated current, with the modulation frequency typically being between 50-100 KHz, and the current magnitude being between 0.1 and 10 mA. Preferably the current pump 102 supplies current with a magnitude of 4 mA that is modulated at 70 kHz through the first electrode 15A. A second electrode 17A injects an identical current (I₂) that is out of phase from I₁ by 180°.

A pair of electrodes 15B, 17B measure the time-dependent voltage encountered by the propagating current. These electrodes are indicated in the figure as V+ and V−. As described above, using Ohm's law (V=I×R), the measured voltage divided by the magnitude of the injected current yields a time-dependent resistance to ac (i.e. impedance) that relates to blood flow in the brachial artery. As shown by the waveform 128 in the figure, the time-dependent resistance features a slowly varying dc offset, characterized by Zo, that indicates the baseline impedance encountered by the injected current; for TBI this will depend, for example, on the amount of fat, bone, muscle, and blood volume in the chest of a given patient. Zo, which typically has a value between about 10 and 150Ω, is also influenced by low-frequency, time-dependent processes such as respiration. Such processes affect the inherent capacitance near the chest region that TBI measures, and are manifested in the waveform by low-frequency undulations, such as those shown in the waveform 128. A relatively small (typically 0.1-0.5Ω) ac component, ΔZ(t), lies on top of Zo and is attributed to changes in resistance caused by the heartbeat-induced blood that propagates in the brachial artery, as described in detail above. ΔZ(t) is processed with a high-pass filter to form a TBI signal that features a collection of individual pulses 130 that are ultimately processed to ultimately determine stroke volume and cardiac output.

Voltage signals measured by the first electrode 15B (V+) and the second electrode 17B (V−) feed into a differential amplifier 107 to form a single, differential voltage signal which is modulated according to the modulation frequency (e.g. 70 kHz) of the current pump 102. From there, the signal flows to a demodulator 106, which also receives a carrier frequency from the current pump 102 to selectively extract signal components that only correspond to the TBI measurement. The collective function of the differential amplifier 107 and demodulator 106 can be accomplished with many different circuits aimed at extracting weak signals, like the TBI signal, from noise. For example, these components can be combined to form a ‘lock-in amplifier’ that selectively amplifies signal components occurring at a well-defined carrier frequency. Or the signal and carrier frequencies can be deconvoluted in much the same way as that used in conventional AM radio using a circuit that features one or more diodes. The phase of the demodulated signal may also be adjusted with a phase-adjusting component 108 during the amplification process. In one embodiment, the ADS 1298 family of chipsets marketed by Texas Instruments may be used for this application. This chipset features fully integrated analog front ends for both ECG and impedance pneumography. The latter measurement is performed with components for digital differential amplification, demodulation, and phase adjustment, such as those used for the TBI measurement, that are integrated directly into the chipset.

Once the TBI signal is extracted, it flows to a series of analog filters 110, 112, 114 within the circuit 100 that remove extraneous noise from the Zo and ΔZ(t) signals. The first low-pass filter 1010 (30 Hz) removes any high-frequency noise components (e.g. power line components at 60 Hz) that may corrupt the signal. Part of this signal that passes through this filter 110, which represents Zo, is ported directly to a channel in an analog-to-digital converter 120. The remaining part of the signal feeds into a high-pass filter 112 (0.1 Hz) that passes high-frequency signal components responsible for the shape of individual TBI pulses 130. This signal then passes through a final low-pass filter 114 (10 Hz) to further remove any high-frequency noise. Finally, the filtered signal passes through a programmable gain amplifier (PGA) 116, which, using a 1.65V reference, amplifies the resultant signal with a computer-controlled gain. The amplified signal represents ΔZ(t), and is ported to a separate channel of the analog-to-digital converter 120, where it is digitized alongside of Zo. The analog-to-digital converter and PGA are integrated directly into the ADS1298 chipset described above. The chipset can simultaneously digitize waveforms such as Zo and ΔZ(t) with 24-bit resolution and sampling rates (e.g. 500 Hz) that are suitable for physiological waveforms. Thus, in theory, this one chipset can perform the function of the differential amplifier 107, demodulator 108, PGA 116, and analog-to-digital converter 120. Reliance of just a single chipset to perform these multiple functions ultimately reduces both size and power consumption of the TBI circuit 100.

Digitized Zo and ΔZ(t) waveforms are received by a microprocessor 124 through a conventional digital interface, such as a SPI or I2C interface. Algorithms for converting the waveforms into actual measurements of SV and CO are performed by the microprocessor 124. The microprocessor 124 also receives digital motion-related waveforms from an on-board accelerometer, and processes these to determine parameters such as the degree/magnitude of motion, frequency of motion, posture, and activity level.

As described above, a Database collects and stores information from the EP procedure and body-worn Telemetry Monitor. FIG. 7 shows examples of simple data fields within the Database 110. In embodiments, for example, the Database 110 includes a high-level, custom schema 109 that describes relationships between data, patients, clinicians, and hospitals. For example, in embodiments the custom schema 109 groups certain hospitals together which have agreed to share data collected from their respective patients, and also groups clinicians within the hospitals who have privileges to view the data. For research purposes, it will likely be necessary to de-identify these data, e.g. remove personal patient information as per the guidelines set out by the Health Insurance Portability and Accountability Act (HIPAA). De-identification will remove sensitive personal information, but will retain demographics information that is stored in a patient demographics data field 108 featuring simple parameters such as a patient identifier (e.g. number), their gender, date of birth, along with simple biometric parameters such as weight, height, and whether or not the patient has an ID. For example, these data can be organized in standard tables used by commercially available relational databases, such as PostgreSQL, Microsoft SQL Server, MySQL, IBM DB2, and Oracle. Typically the patient identifier within the patient demographics field 108 is a database ‘key’ that links a particular patient to other data fields. For example, other data fields within the database 110, such as the pre-procedure 106, in-procedure 104, and post-procedure 103 data fields, use this key to link physiological data measured during these particular periods to the patient. These data are found in new tables 118a-c in the database, and typically include physiological data (e.g. numerical values and waveforms) describing parameters such as HR, systolic and diastolic blood pressure (BP), respiratory rate (RR), and blood oxygen (SpO2). Typically these parameters are measured over time (e.g. in a continuous or quasi-continuous manner), and then identified in the tables 118a-c by a ‘Run’ number that sequentially increases over time. As described above, data for the tables 118a-c is typically measured with a hardware component attached to the patient, such as the Telemetry Monitor that an ambulatory patient wears outside of the hospital, an ID, or by a VS monitor used to measure the patient during an actual EP procedure.

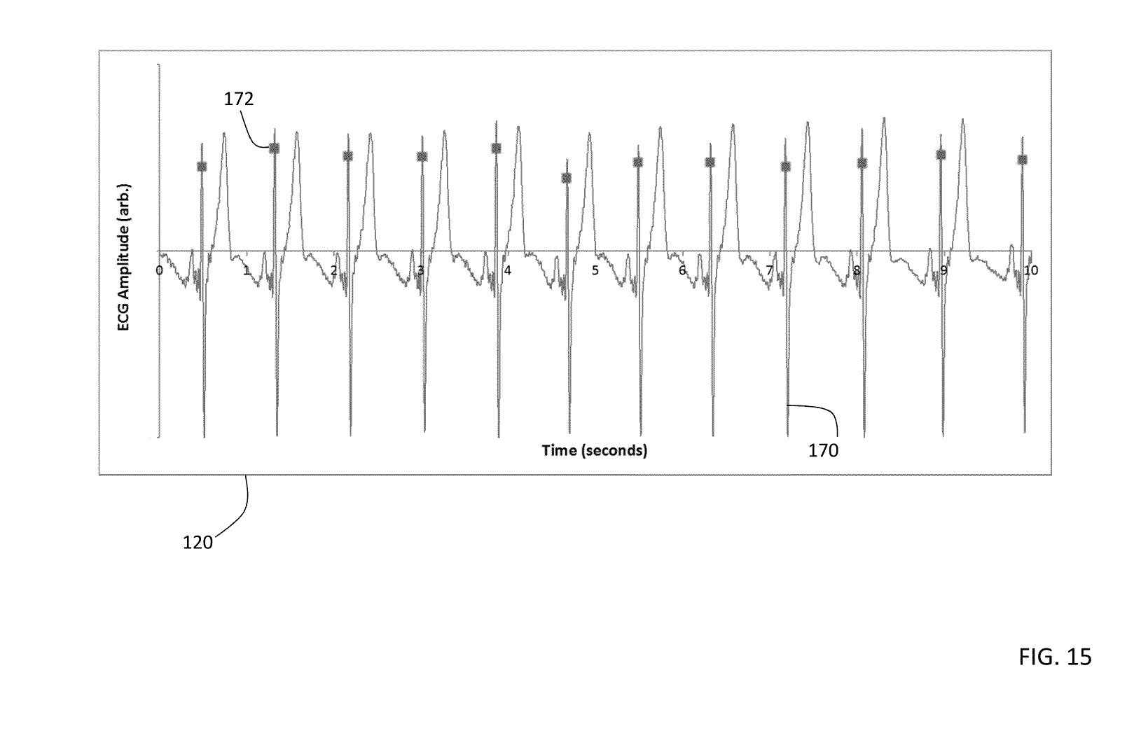

The database may also associate numerical physiological data for each run with a physiological waveform 120a-c that is analyzed to extract the particular datum. For example, as shown below in FIG. 15, the above-mentioned hardware component may measure time-dependent ECG waveforms 120a-c that yield information such as HR and arrhythmia information, and are thus stored in the database. Such waveforms may be processed with the algorithm-based tools, such as numerical ‘fitting’ or beatpicking algorithms, to better diagnose a patient's condition. Although FIG. 15 only shows single-lead ECG waveforms, other physiological waveforms can also be measured, stored, and then processed with the algorithm-based tools described above. These waveforms include multi-lead ECG waveforms, TBI waveforms, and photoplethysmogram (PPG) waveforms that yield SpO2. In embodiments, these waveforms may be associated with another table that includes annotation markers that indicate fiducial points (e.g., the QRS complex in an ECG waveform) associated with certain features in the waveforms. The algorithm-based tools may also process these annotation markers to perform simple patient follow-up, estimate patient outcomes, and do applied and academic research, as described above.

In related embodiments, ECG waveforms may be analyzed with more complex mathematical models that attempt to associate features of the waveforms with specific bioelectric events associated with the patient. For example, mathematical models can be deployed that estimate ECG waveforms by interactively changing the estimated timing associated with depolarization and repolarization of a simulated ventricular surface, as well as the strength of the depolarization and repolarization. The timings and signal strengths associated with these models can then be collectively analyzed to simulate an ECG waveform. The simulated ECG waveform can then be compared to the waveform actually measured from the patient to help characterize their cardiac condition, or the efficacy of the EP procedure that addresses this condition. In general, a wide range of physiological and device-related parameters can be stored in the data tables described above. Examples of some of these data fields corresponding to specific ECP procedures are shown below in Table 1.

In embodiments, commercially available software tools, such as Mortara's E-Scribe Rx and VERITASÔ ECG algorithms, may be interfaced with the database 110 and used to analyze ECG waveforms measured from the patient. These software tools are designed to analyze complex, multi-lead ECG waveforms to determine complex arrhythmias, VF, VT, etc.

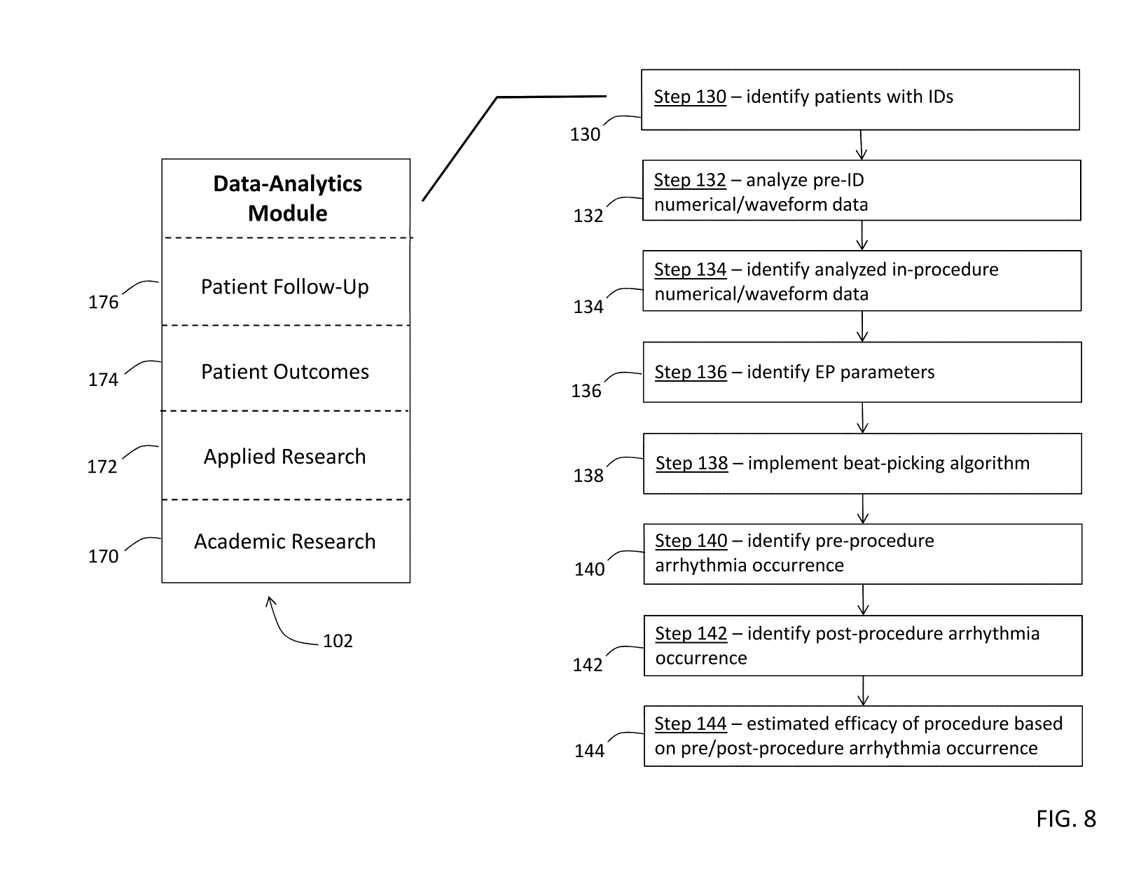

FIG. 8 shows a simple example of a simple Data Analytics System 102 featuring an algorithm-based tool that analyzes patient data from the data-collection/storage module to estimate a patient's outcome. In one specific algorithm associated with the Data Analytics System 102, computer code analyzes data fields to first identify patients with IDs (step 130). The code then collects pre-ID (step 132) and in-procedure (step 134) numerical/waveforms data, along with parameters from the patient's EP procedure (step 136), and readies them for analysis. Parameters collected during the patient's EP procedure include parameters associated with the EP catheter used during the EP procedure (such as those described in Table 1), potentials applied by the catheter and their timing, and two and three-dimensional images measured during the procedure. The algorithm then collectively analyzes these data, and implements a beat-picking algorithm (step 138) to further characterize ECG waveforms measured during steps 134 and 136. The beat-picking algorithm can determine parameters such as induced arrhythmia, effective refractory periods, characteristics of specific components within the patient's ECG waveform, e.g. the QRS complex, width of the P-wave, QT period and dispersion, and instantaneous HR.

Using these technologies, the algorithm can perform simple functions like identifying pre-procedure (step 140) and post-procedure (step 142) arrhythmia occurrences, and then comparing these to determine the efficacy of the procedure (step 144). Many other algorithm-based tools, of course, are possible within the scope of this invention.

Other algorithm-based tools are more sophisticated than that described with reference to FIG. 8. In general, these tools can analyze any combination of data that are generated by the systems described above.

| Description of | # of Possible | |
|---|---|---|
| Data Field | Values | Example Values |
| Ablated | 35 | AV Node Modification (Fast pathway), Bundle |
| Locations | | Branch, Complex fracionated atrial electrograms |
| | | (CFAE), Crista Terminalis, LA Anteroseptal line, LA |
| | | CS Line, Left atrium, RIGHT ATRIUM, Accessory |
| | | Pathway, AV Node, Cavo-tricuspid isthmus, |
| | | Endocardial, Epicardial, Fast pathway, Intermediate |
| | | pathway, LEFT CIRCUMFERENTIAL |
| | | PULMONARY, Segmental antral left lower |
| | | pulmonary vein, Segmental antral left lower |
| | | pulmonary vein, MITRAL ISTHMUS, Fast pathway, |
| | | Left Atrial Linear (Mitral Isthmus), Right |
| | | Circumferential Pulmonary, Left Atrial Linear (Mitral |
| | | Isthmus), Endocardial, Right Circumferential |
| | | Pulmonary, EPICARDIAL, Segmental antral right |
| | | lower pulmonary vein, Left Atrial Linear (Roof), |
| | | Segmental antral right upper pulmonary vein, |
| | | Segmental antral right upper pulmonary vein, SVC, |
| | | Slow pathway, Segmental antral right lower |
| | | pulmonary vein. |
| Sub-Locations | 106 | Left Circle, LV Septal Basal, CS middle, Lower crista, |
| | | LA septal wall, Mitral Valve Annulus, RA lateral wall, |
| | | Left Antero-Lateral, Non-Coronary Cusp, Upper |
| | | crista, RVOT Anterior, LA Scar, Atrio-Ventricular, |
| | | Left Lateral, Right Mahaim, CS proximal, Atrio- |
| | | Fasicular, Right Mid-Septal, RV Posterior Basal, CS |
| | | distal, LA appendage, LA anterior wall, Lower Loop, |
| | | Left Aortic Cusp, LV anterior Fascicle, LA septum, |
| | | RV Anterior Apical, LV Posterior Mid, LV Posterior |
| | | Fascicle, LV Posterior Apical, RV Anterior Mid, |
| | | LLPV, RLPV, RVOT Free Wall, RV Septal Apical, |
| | | RV Lateral Mid, Mitral Isthmus (with CS), Right |
| | | Postero-Lateral, RBB, LV Lateral Basal, Left Antero- |
| | | Septal, RA septal wall, LV Septal Apical, MVA |
| | | anterior, LV Outlow Tract, Upper Loop, Pulmonary |
| | | Artery, Right Antero-Lateral, TVA lateral, Right |
| | | Aortic Cusp, RA Scar, Right Posterior, RA anterior |
| | | wall, Mitral Isthmus (endocardial only), RV Posterior |
| | | Apical, CSos, LV Anterior Mid, RV Lateral Basal, |
| | | Left Mahaim, TVA posterior, RA poseterior wall, |
| | | Nodo-Fasicular, LV Lateral Mid, RA appendage, |
| | | Cavo-Tricuspid Isthmus, LA lateral wall, RVOT |
| | | Posterior, Middle crista, Superior Vena Cava, Left |
| | | Posterior, LV Anterior Basal, Fossa ovalls, LV Septal |
| | | Mid, LUPV, Diverticular, Diverticuar, SVC, Non- |
| | | Coronary Aortic Cusp, TVA anterior, Right Lateral, |
| | | RVOT Septal, MVA septal, RUPV, LA posterior wall, |
| | | Right Postero-Septal, MVA posterior, Nodo- |
| | | Ventricular, MVA lateral, RV Anterior Basal, LV |
| | | Lateral Apical, Left Postero-Septal, Right Antero- |
| | | Septal, LVOT, RV Septal Mid, Left Postero-Lateral, |
| | | RV Septal Basal, LA roof, Left bundle branch, LA |
| | | poseterior wall, RV Posterior Mid, RA septum, RV |
| | | Outflow Tract Anterior, RV Lateral Apical, Csos, LV |
| | | Posterior Basal, Right Circle |
| Access | 29 | Left Subclavian Vein, Right Antecubital Vein, Right |
| Locations | | Femoral Vein, Right Subclavian Vein, Right Lower |
| | | Extremeties/Thigh, Left Antecubital Vein, Superficial |
| | | Right Leg, Superficial Right Hand/Forearm Vein, |
| | | Deep Right Hand/Forearm Vein, Right Femoral |
| | | Artery, Superficial Right Arm Vein, Superficial Left |
| | | Hand/Forearm Vein, Deep Right Arm Vein, Deep |
| | | Right Arm Vein, Deep Left Hand/Forearm Vein, Left |
| | | Femoral Vein, Left Lower Extremeties/Thigh, Right |
| | | Foot, Right Internal Jugular Vein, Superficial Left |
| | | Leg, Deep Right Leg, Left Femoral Artery, Left |
| | | Internal Jugular Vein, Deep Left Arm Vein, Left |
| | | Radial Artery, Right Radial Rrtery, Superficial Left |
| | | Arm Vein, Left Foot, Deep Left Leg |
| Arrhythmia | 20 | Idiopathic ventricular tachycardia, Atrial Fibrillation |
| Mechanism | | Paroxysmal, AV Nodal Reentry (fast-slow), AV Nodal |
| | | Reentry (slow-slow), Premature ventricular |
| | | contractions, Atrial Fibrillation Persistent, Atypical |
| | | Left Atrial Flutter, Atypical Mitral Isthmus Flutter, |
| | | Bundle Branch Reentry VT, Inappropriate Sinus |
| | | Tachycardia, Structural ventricular tachycardia - |
| | | Dilated Cardi, AV Nodal Reentry (slow-fast), Focal |
| | | Atrial Tachycardia, Antidromic AV reentrant |
| | | tachycardia, Reverse Typical Atrial Flutter, Atypical |
| | | Right Atrial Flutter, Typical Atrial Flutter, Structural |
| | | ventricular tachycardia - Ischemic Card, Wolff- |
| | | Parkinson-White syndrome, Orthodromic AV |
| | | reentrant tachycardia |
| Arrhythmia | 10 | Typical Atrial Flutter, AV nodal reentry (slow-slow), |
| Mechanism | | AV nodal reentry (slow-fast), Antidromic AV |
| Types | | reentrant tachycardia (ART), Reverse Typcial Atrial |
| | | Flutter, Ventricular tachycardia, Orthodromic AV |
| | | reentrant tachycardia (ORT), Atrial Fibrillation, |
| | | Atypical Atrial Flutter, AV nodal reentry (fast-slow) |
| Arrhythmia | 9 | Vagal Effect, Arrhythmogenic Veins RUPV, |
| Observations | | Arrhythmogenic Veins LLPV, Concealed Accessory |
| | | Pathway, Negative CSM, WPW, Positive CSM, |
| | | Arrhythmogenic Veins LUPV, Arrhythmogenic Veins |
| | | RLPV |
| Axis Deviations | 6 | Left, Left Inferior, None, Right Inferior, Right, Left |
| | | Superior |
| Mapping | 8 | Carto 3D electro-anatomical, Fluoroscopy, Ensite 3D |
| Systems | | Balloon Array, ESI NavX 3D electro-anatomical |
| Energy Sources | 6 | Cryoablation, Laser, Ultrasound, Other, |
| | | Radiofrequency |
| Morphology | 8 | |
| Pacing Site | 13 | LVA, LRA, LA, RVOT, RVA, LVB, CSP, CSP, LLA, |
| | | HRA, CSD, CSM, LVOT |
| lu_abl_result | 51 | Intermediate pathway block - not reinducible, Partially |
| | | Isolated, ORT Reinducible, Right bundle branch |
| | | block, AV Node Block, AV Node Modified, Fast |
| | | pathway block - not reinducible, VT Not-reinducible, |
| | | Conduction Block, Isolated, AVNRT Reinducible, |
| | | Mitral Isthmus Block (bidirectional), ORT Not |
| | | Reinducible, Bidirectional CTI Block, AFL |
| | | Terminated, PVCs eliminated, LLPV Isolated, Left |
| | | bundle branch block, VT Slowed, WPW Terminated, |
| | | FAT terminated, ORT Terminated, Reduction in |
| | | electrogram amplitude to less than 0.5 mV, RMPV |
| | | Isolated, AP block, not reinducible, RUPV Isolated, |
| | | AF Terminated, Complete AV Block, Slow pathway |
| | | block - not reinducible, AF Converted to AFL, AFL |
| | | Not Reinducible, AP Block, Reduction in electrogram |
| | | amplitude to less than 0., VT Terminated, Mitral |
| | | Isthmus Conduction Delay Only, LUPV Isolated, |
| | | Single AV nodal echo only, ART Reinducible, AF |
| | | Termination, AP Block, Not Reinducible, ART Not |
| | | Reinducible, ART Terminated, WPW Reinducible, |
| | | Mitral Isthmus Block (unidirectional), CTI conduction |
| | | delay, Incomplete AV Block, Mitral Isthmus |
| | | Conduction Delay, AP block (antegrade and |
| | | retrograde), RLPV Isolated, AP block (antegrade |
| | | only), Unidirectional CTI Block |
| Structural | 8 | Atrial Septal Defect, Patent Foramen Ovale, Common |
| Observations | | OS Left, Atrial Scarring, LA Thrombus, Common OS |
| | | Right, Pericardial Effusion |
| Termination | 11 | Cardioversion, Ablation, Burst, Verapamil, |
| Methods | | Adenosine, Spontaneous, Metropolol, Pvc, |
| | | Procainamide, Ibutilide, Pac |
| Access Type | 21 | Direct Cutdown, Percutaneous, Epicardial, Swan-Ganz |
| | | Line, Tunneled Central Line, Arterial Line, Central |
| | | Venous Pressure Line, Sheath - Hansen, Sheath - |
| | | Trans septal, Peripherally Inserted Central Catheter, |
| | | Pulmonary Artery Catheter, Shunt, Sheath - Steerable, |
| | | Sheath - Standard short, Sheath - Preformed long, |
| | | Central Venous Line, Peripheral IV, Implantable Port |

Table 1—Data Fields Associated with Specific EP Procedures

FIGS. 9 and 10 show examples of user interfaces 190, 191, 192, 193 that integrate with the above-mentioned systems and run on an iPhone 20 and Android tablet 21. The user interface show information such as patient demographics (interface 190), patient-oriented messages (interface 191), and numerical vital signs and time-dependent waveforms (interfaces 192, 193). The interfaces shown in the figures are designed for the clinician. More screens, of course, can be added, and similar interfaces (preferably with less technical detail) can be designed for the actual patient. The interfaces can also be used to render operational reports, such as the report 193 shown in FIG. 11. This report indicates the number and type of EP procedures performed by clinicians at a given hospital. Reports showing similar data are, of course, possible.

FIG. 11 shows an example report 193 from the data-analytics module. The report 193, for example, could be taken from a GUI of a website. It features four ‘areas’ of analytics that, informally, vary in terms of their complexity. In the upper left-hand corner, the report 193 includes a bar chart that shows the number of EP procedure conducted on a monthly basis. The upper right-hand corner shows a monthly breakdown of EP procedures performed in different procedure rooms, i.e. EP labs. The lower right-hand corner shows a monthly breakdown of different types of EP procedures. And the lower left-hand corner shows a monthly breakdown of nurses participating in the various EP procedures.

A variety of other reports are possible with the system described herein. For example, the above-mentioned system can be used to generate clinical analyses and subsequent reports for the clinician that include the following information:
1—physiological information before and after EP treatment
2—ECG and TBI waveforms and their various components before and after treatment
3—estimated efficacy of EP treatment
4—the need for EP treatment
5—correlation of patient demographics and EP efficacy
6—correlation of physiological information and EP efficacy
7—correlation between ablation characteristics (e.g. ablation potentials, locations) and stabilization of cardiac rhythm
8—efficacy of ID/leads and stabilization of cardiac rhythm
9—ID battery voltage and stabilization of cardiac rhythm
10—correlation between heart rate variability and occurrence of cardiac trauma (e.g. stroke, myocardial infarction) within well-defined periods of time

Other clinical analyses are made possible with the invention described here, and are thus within its scope.

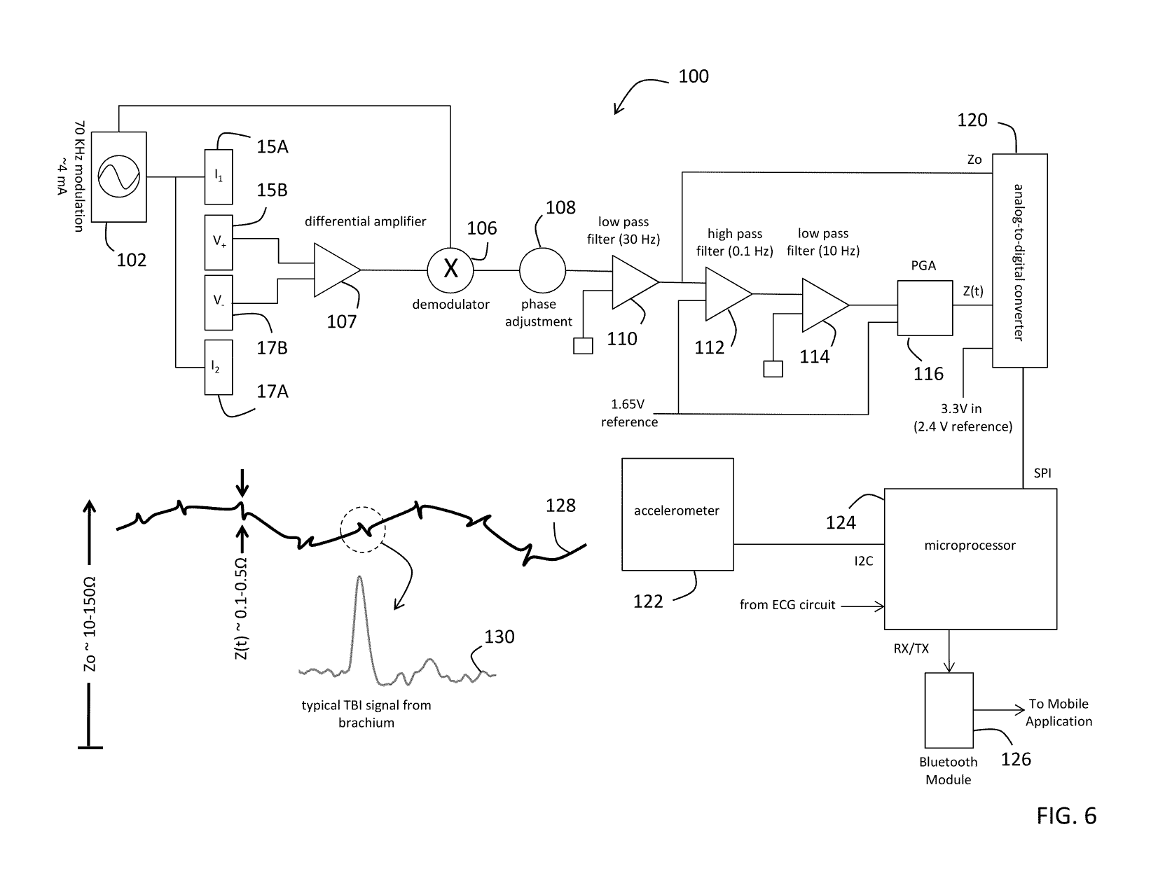

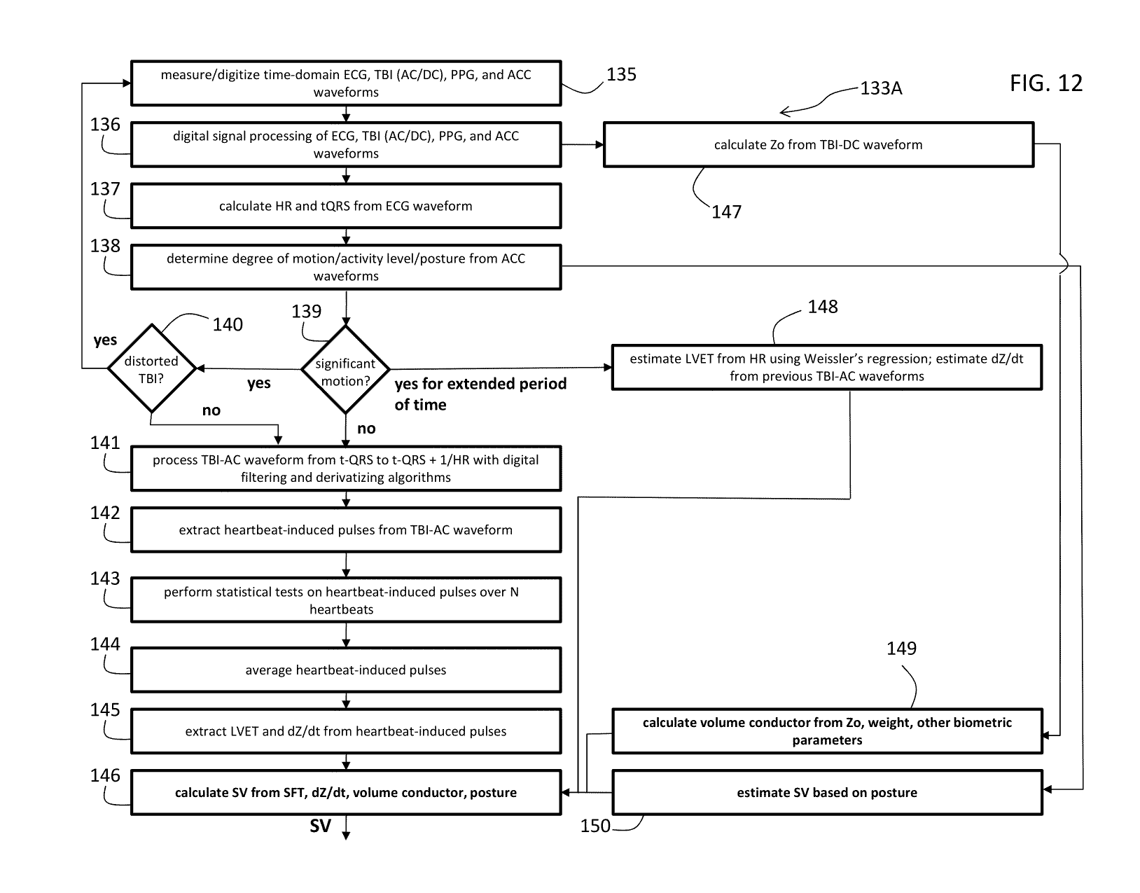

FIG. 12 shows a flow chart of an algorithm 133A that functions using compiled computer code that operates, e.g., on the microprocessor 124 shown in FIG. 6. The compiled computer code is loaded in memory associated with the microprocessor, and is run each time a TBI measurement is converted into a numerical value for CO and SV. The microprocessor typically runs an embedded real-time operating system. The compiled computer code is typically written in a language such as C, C++, or assembly language. Each step 135-150 in the algorithm 133A is typically carried out by a function or calculation included in the compiled computer code.

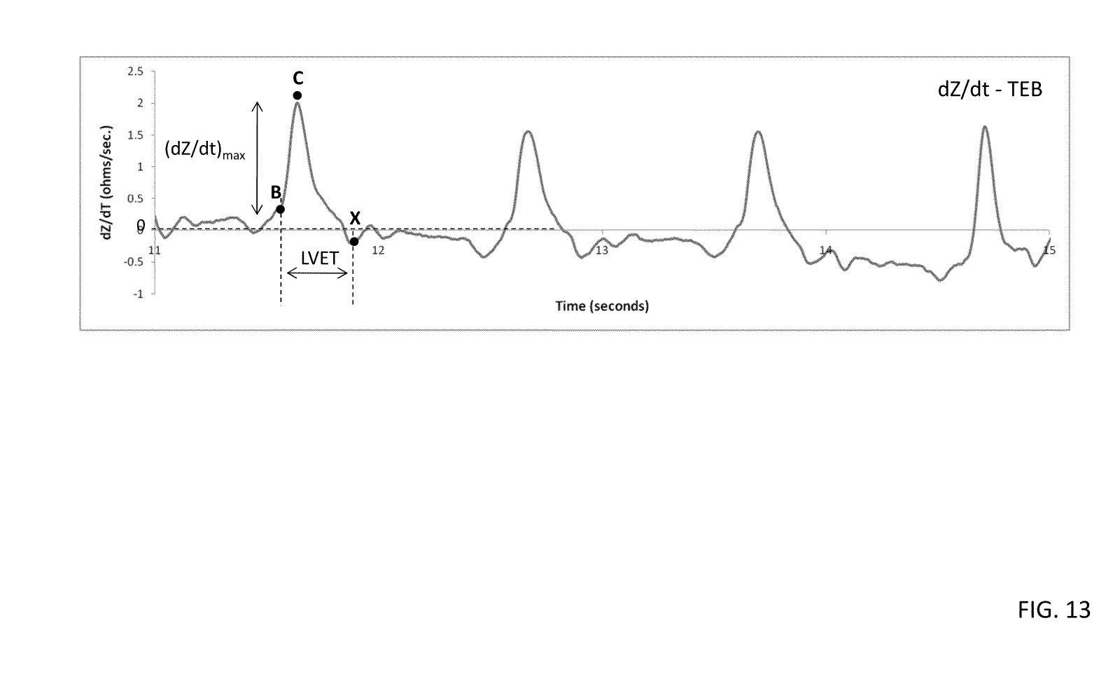

FIG. 13 indicates how LVET is extracted from the derivatized TBI waveform. The derivatized ICG waveform features consecutive pulses, each characterized by three points: a ‘B’ point on the pulse's upswing indicating opening of the aortic valve; an X point on the pulse's nadir indicating closing of the aortic valve; and a ‘C’ point on its maximum value indicating the maximum slope of the ΔZ(t) pulse's upswing, which is equivalent to (dZ/dt)ₘₐₓ. LVET is typically calculated from the time differential between the B and X points. However, due to the subtle nature of these fiducial markers, even low levels of noise in the waveforms can make them difficult to determine. Ultimately such noise adds errors to the calculated LVET and resulting SV.

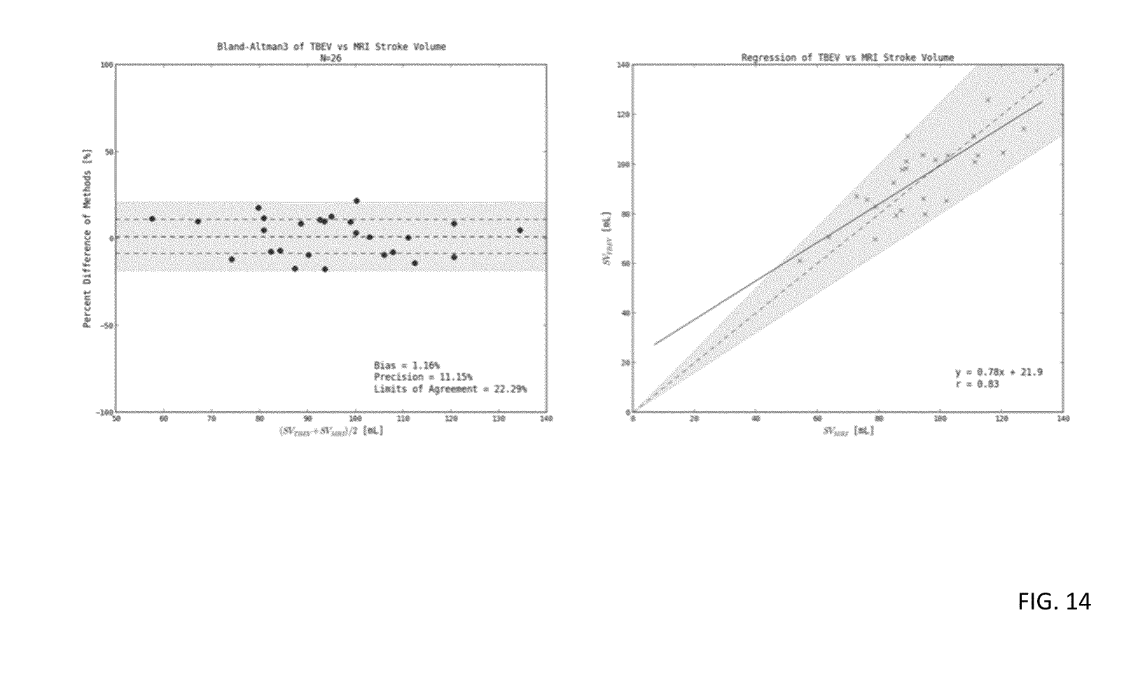

The analysis described above was used in a formal clinical study to test accuracy of determining SV using a technique similar to TBI and Eq. 2 above, compared to CO determined using MRI. The device used to measure TBI had a form factor similar to that shown in FIG. 3. Correlation and Bland-Altman plots are shown, respectively, in the right and left-hand sides of FIG. 14. The shaded gray area in the plots indicates the inherent errors associated with conventional Doppler/ultrasound measurements, which are about +/−20%. In total 26 subjects (14M, 12W) with ages ranging from 21-80 were measured for this study, and correlations for all of these subjects fell within the error of the MRI measurements.

FIG. 15 shows an example of an ECG waveform 170 that is measured from a patient (e.g., before the EP procedure), stored in the database, and then analyzed by an algorithmic-based tool such as that described with reference to FIG. 8 to estimate the patient's cardiac performance. The ECG waveform 170, which in this case corresponds to a relatively healthy patient, features a collection of equally spaced, time-dependent data points that are defined by a sampling rate of an ECG monitor, which in this case is 500 Hz. The waveform features a sharply varying peak, called the QRS complex, which indicates initial depolarization of the heart and informally marks the onset of the patient's cardiac cycle. Each heartbeat yields a new QRS complex. After a few hundred milliseconds, a relatively slowly varying feature called the T-wave follows the QRS complex. In general, each patient features a unique ECG waveform from which the algorithmic-based tools can extract important cardiac information. As described above with reference to FIG. 8, a simple algorithmic-based tool called a ‘beatpicker’ analyzes the ECG waveform 170 to determine the patient's HR and arrhythmia information. In this application, the beatpicker uses an algorithm (called the Pan-Thompkins algorithm) that determines the temporal location of the QRS complex corresponding to each heartbeat. The Pan-Thompkins algorithm typically includes the following steps: i) filtering the ECG waveform to remove any high-frequency noise; ii) taking a mathematical derivative of the waveform; iii) squaring the waveform; iv) signal averaging the waveform; and v) finding the peaks of the waveform processed with steps i)-iv). Locations of the QRS complex from waveforms processed in this manner are shown in the figure by a collection of gray squares 172. Once the collection of QRS complexes is located, the algorithmic-based tool can determine the patient's HR and arrhythmia information using well-known techniques in the art.

The ECG waveform 170 described above is relatively simple, and other than a relatively tall T-wave, lacks any complicated features that challenge conventional beatpickers. However, such features are not uncommon amongst cardiac patients, and thus the beatpicker must be sophisticated enough to analyze them. Moreover, the ECG waveform 170 shown in FIG. 15 only corresponds to a single lead, and thus is relatively unsophisticated and lacks information describing complex cardiovascular performance. Typically, the system according to this invention analyzes multi-lead ECG waveforms. Multi-lead ECG waveforms can contain information from 5, 7, and even 12-lead ECGs. In general, these types of ECG waveforms are required to evaluate the complex cardiovascular performance associated with patients that would most benefit from the present invention.

For example, in embodiments, algorithmic-based tools according to the invention, or software associated with these tools, can also analyze relatively long traces of ECG waveforms (spanning over seconds or minutes) measured before, during, and after the EP procedure to characterize: i) a given patient; ii) the efficacy of the EP procedure applied to that patient; iii) a given patient's need for an EP procedure; or iv) the overall efficacy of the EP procedure as applied to a group of patients. Analysis of the relatively long traces of ECG waveforms in this manner may indicate cardiac conditions such as cardiac bradyarrhythmias, blockage of an artery feeding the heart, acute coronary syndrome, advanced age (fibrosis), inflammation (caused by, e.g., Lyme disease or Chaga's disease), congenital heart disease, ischaemia, genetic cardiac disorders, supraventricular tachycardia such as sinus tachycardia, atrial tachycardia, atrial flutter, atrial fibrillation, junctional tachycardia, AV nodal reentry tachycardia and AV reentrant tachycardia, reentrant tachycardia, Wolff-Parkinson-White (WPW) Syndrome, Lown-Ganong-Levine (LGL) Syndrome, and ventricular tachycardia Likewise, analysis of these cardiac conditions by analyzing the ECG waveforms may indicate the efficacy of the EP procedure.

Typically, before the algorithmic-based tool deploys the beatpicker, it is analyzed against well-known databases, such as the MIT arrhythmia database or the American Heart Association database, to determine its performance. Beatpickers with a performance of about 95% or greater, as evaluated relative to these standards, are typically categorized as acceptable. Alternatively, as described above, the algorithm-based tools may integrate with commercially available tools for analyzing ECG waveforms, such as those developed and marketed by Mortara.

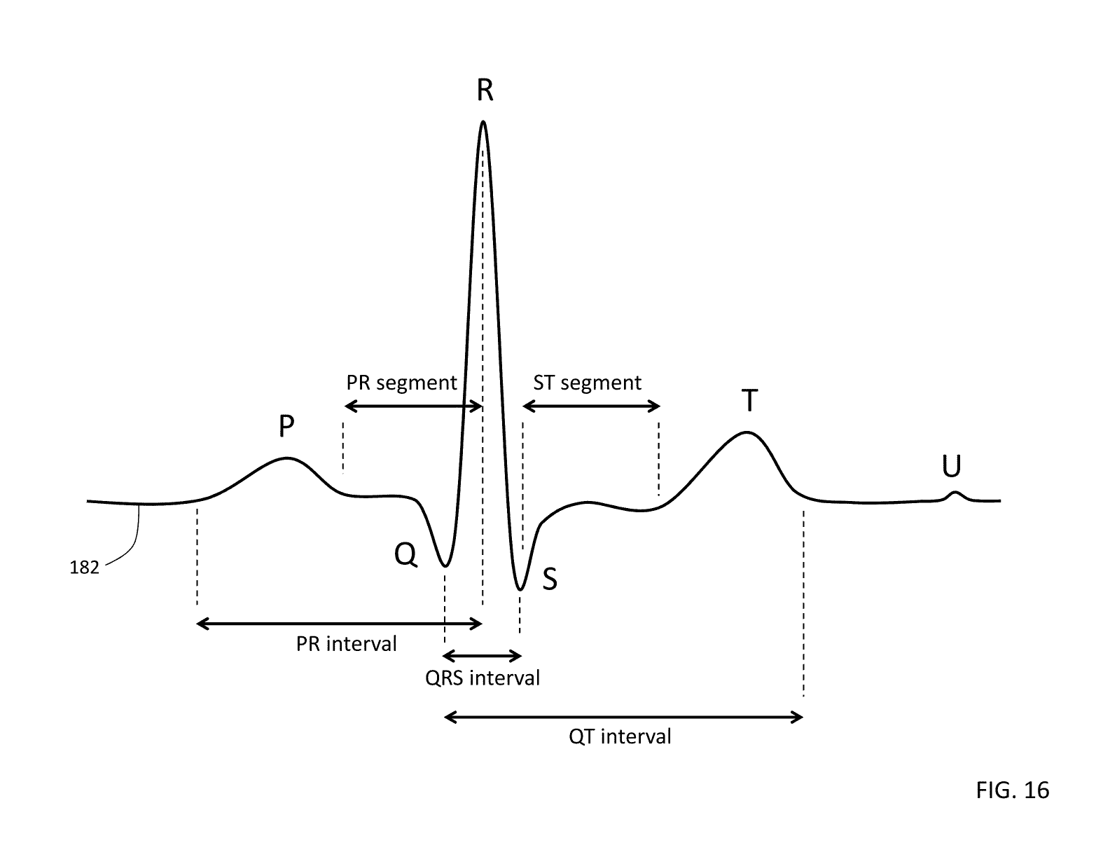

FIG. 16 shows a waveform snippet 182 found within the ECG waveform 170 that is shown in FIG. 15. The waveform snippet 182 corresponds to a single heartbeat. Waveform snippets 182 may be collected before, during, and after an EP procedure, and are typically analyzed after they are stored in the database, as described above. Algorithm-based tools within the system, or software components within the algorithm-based tools, may analyze one or more waveform snippets 182 generated by a given patient to predict certain cardiac conditions assigned to that patient. Alternatively, the software may collectively analyze waveform snippets corresponding to large groups of patients to evaluate, e.g., the efficacy of a certain aspect of an EP procedure, or predict how a given EP procedure is likely to affect a given patient.

As shown in the figure, the waveform snippet features the following components: i) a QRS complex; ii) a P-wave; iii) a T-wave; iv) a U-wave; v) a PR interval; vi) a QRS interval; vii) a QT interval; viii) a PR segment; and ix) an ST segment. Algorithmic-based tools within the system, or software associated with the algorithm-based tools, can analyze each of these components and their evolution over time as described above. In particular, algorithmic-based tools that perform numerical fitting or pattern recognition may be deployed to determine the components and their temporal and amplitude characteristics for any given heartbeat recorded by the system. Each component corresponds to a different feature of the patient's cardiac system. For example, the PR interval (which typically has a duration between about 120-200 ms) represents the time from firing of the patient's SA node to the end of the delay of their AV node. A prolonged PR interval, or a PR interval that is inconsistent over time, may indicate blockage of an artery feeding the patient's heart. Alternatively, a shortened or non-existent PR interval may indicate a cardiac condition such as tachycardic, junctional, ectopic, or ventricular rhythms. The QRS interval, which is typically between 40-100 ms, represents the travel time of electrical activity through the patient's ventricles and ventricular depolarization that drives contraction of the heart. QRS intervals that are longer than this, or that feature a ‘notch’, can indicate aberrant ventricular activity or cardiac rhythms with a ventricular focus.

Variation in the time between subsequent QRS complexes (i.e., the time associated with a given HR) may also indicate a cardiac condition. In general, some variation in this component is normal and indicative of a healthy heart. Little or no variation, which typically becomes more pronounced as the patient ages, or a sudden decrease in variation, may indicate the onset of a cardiac event.

The QT interval, which is typically less than 50% of the total duration of the time associated with the patient's HR, represents the travel time of electrical activity through the patient's ventricles to the end of ventricular repolarization. This parameter varies with HR, and also with age and gender. Prolonged QT intervals represent a prolonged time to cardiac repolarization, and may indicate the onset of ventricular dysrhythmias.

The P-wave, which proceeds the QRS complex of each heartbeat, is typically upright and uniform in shape, and indicates the firing of the SA node and subsequent atrial depolarization; it typically has a width of about 50 ms, and an amplitude that is about 10-20% of the QRS amplitude. P waves that are abnormally wide or notched, or tall and peaked, indicate cardiac conditions such as P-mitrale and P-pulmonale, respectively. The PR segment, which separates this feature from the QRS complex, is typically 120-200 ms in duration, and represents the delay separating the firing of the SA node and ventricular depolarization. A PR segment that gradually increases over time may indicate the onset of damage to the patient's heart. The T-wave, which follows the QRS complex, indicates the onset of ventricular repolarization, and should appear rounded and somewhat symmetrical; the peak of the T-wave is typically relatively close to the wave's end. T-waves that are abnormally tall or ‘tented’ may indicate cardiac conditions such as hyperkalemia or myorcardial injury. T-waves that are inverted may indicate cardiac conditions such as myocardial ischemia, myocardial infarction, pericarditis, ventricular enlargement, bundle branch block, subarachnoid hemorrhage, and the presence of certain pharmaceutical compounds, such as quinidine or procainamide.

The U-wave, which is somewhat uncommon and when present only about 2-5% of the amplitude of the QRS complex, depicts the last phase of ventricular repolarization. It is typically present with patients undergoing bradycardia, and can be enlarged during cardiac conditions such as hypokalemia, cardiomyopathy, or enlargement of the left ventricle.

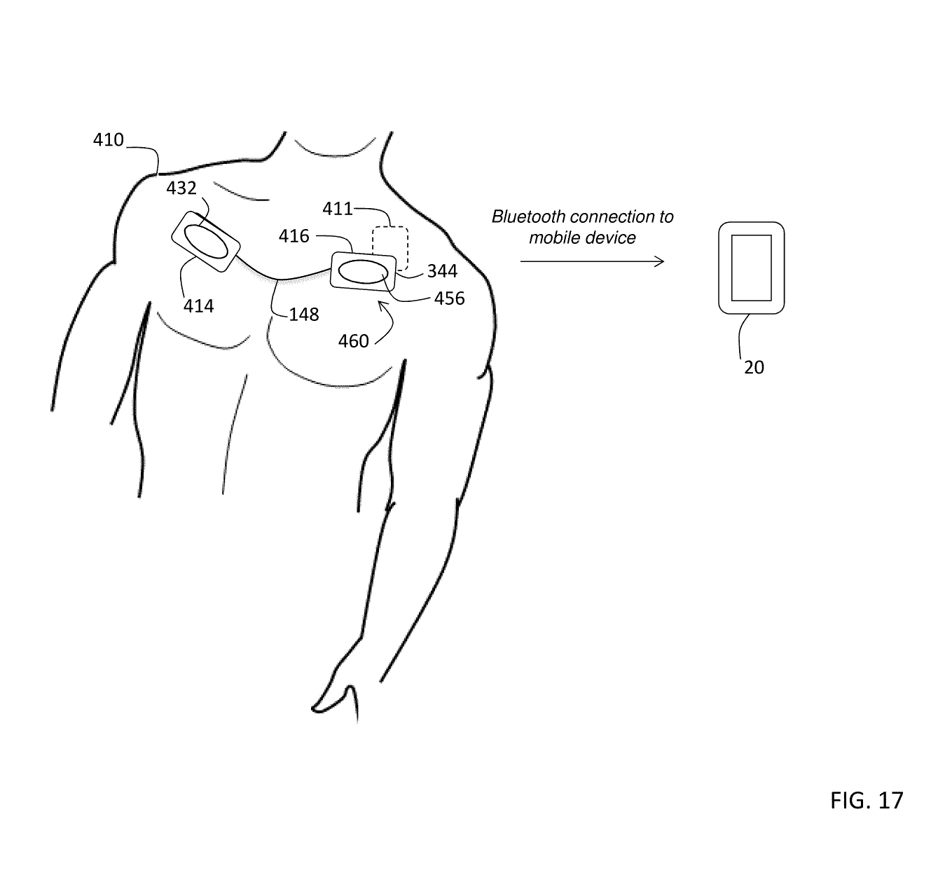

TBI, like techniques such as impedance pneumography, injects small amounts of current into the patient's body, and measures resistance (i.e. impedance) encountered by the current to calculate a parameter of interest. During a TBI measurement, heartbeat-induced blood flow results in the pulsatile component of ΔZ(t). Additionally, changes in capacitance due to breathing may also affect the impedance as measured by TBI. FIGS. 17A-C illustrate this point. In FIG. 17A, for example, a TBI waveform with no digital filtering shows both high-frequency cardiac components due to blood flow, as well as low-frequency undulations due to respiration rate. Both features can be extracted and analyzed using digital filtering. For example, as shown in FIG. 17B, processing the TBI waveform shown in FIG. 17A with a first band-pass filter (0.5→15 Hz) removes the respiratory component, leaving only the cardiac component. Similarly, as shown in FIG. 17C, processing the TBI waveform shown in FIG. 17A with a second band-pass filter (0.001→1 Hz) removes the cardiac component, leaving on the undulations due to respiration. In this latter case, the peaks in the waveform can be counted with a conventional breath-picking algorithm to determine respiration rate.

Other embodiments are also within the scope of the invention. For example, other techniques besides the above-described algorithms can be used to analyze data collected with the system. Additionally, processing units and probes for measuring ECG waveforms similar to those described above can be modified and worn on other portions of the patient's body. For example, the ECG-measuring system can be in a patch configuration. Or they can be modified to attach to other sites that yield ECG waveforms, such as the back or arm. In these embodiments the processing unit can be worn in places other than the wrist, such as around the neck (and supported, e.g., by a lanyard) or on the patient's waist (supported, e.g., by a clip that attaches to the patient's belt). In still other embodiments the probe and processing unit are integrated into a single unit. In still other embodiments, the systems for measuring ECG waveforms are implanted or inserted in the patient, e.g. they are part of the ID or EP system.

Systems similar to that described above can also be used for other cardiac procedures conducted in other areas of the hospital, such as the catheterization laboratory, medical clinic, or vascular analysis laboratory. In these applications, data other than HR and ECG waveforms may be analyzed using techniques similar to those described above. Data used in these examples includes medical images (such as those measured using MRI or Doppler/ultrasound), all vital signs, hemodynamic properties such as cardiac output and stroke volume, tissue perfusion, pH, hematocrit, and parameters determined with laboratory studies.

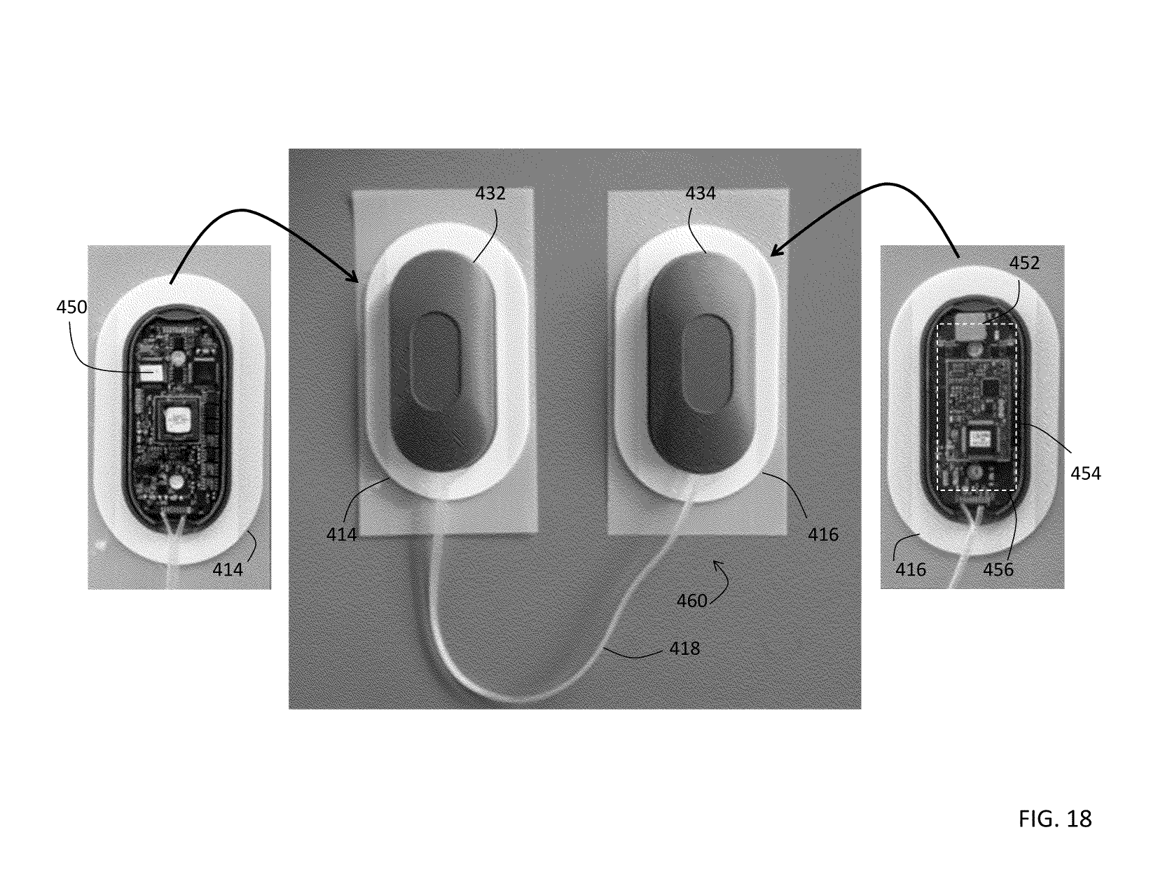

FIGS. 17 and 18 show an alternative embodiment of the invention. Here, the body-worn monitor includes the same components as those referenced with respect to FIGS. 2 and 3, and additionally includes a ‘reader circuit’ 456 that reads information from an ID 411, such as a pacemaker or implantable cardioverter defibrillator. Typically these devices are implanted near the patient's shoulder on their left-hand side, as shown in FIG. 17. The reader circuit typically operates using radio frequencies in either the Industrial, Scientific and Medical (ISM) band (e.g. from 902-928 MHz) or a subsection of the Medical Implant and Communications (MICS) band (e.g. from 402-405 MHz). To accomplish this, the reader circuit 456 typically features a circuit for inductive magnetic coupling, which is similar to that used in the ‘wands’ of most ID interrogators (often called ‘programmers’). Alternatively, the reader circuit 456 can be a short-range wireless radio. In both cases, the reader circuit reads wirelessly transmitted diagnostic data stored in memory within the ID, and then stores these data in memory associated with the microprocessor for later use. Typically the data are uploaded as an encrypted data, and then decoded by the microprocessor.

Still other embodiments are within the scope of the following claims.

What is claimed is:

1. A system for characterizing a patient, comprising:
a data-processing software system that interfaces to a treatment software system and a body-worn monitor, the data-processing software system configured to analyze data collected during and after an invasive cardiac treatment program, the treatment software system configured to collect data during the invasive cardiac treatment program, and the body-worn monitor configured to measure heart rate (HR) and stroke volume (SV) from the patient after the cardiac treatment program and transmit this to the data-processing software system, which then modifies the measurement of SV using the data collected during the cardiac treatment program.

2. The system of claim 1, wherein the treatment software system configured to collect data describing SV during the invasive cardiac treatment program.

3. The system of claim 2, wherein the data describing SV is measured with a pulmonary arterial catheter.

4. The system of claim 2, wherein the data describing SV collected during the cardiac treatment program is used to calibrate the measurement of SV made by the body-worn monitor.

5. The system of claim 2, wherein the data-processing software system uses a linear regression algorithm that processors the SV collected during the invasive cardiac treatment program to calibrate the measurement of SV made by the body-worn monitor.

6. The system of claim 1, wherein the body-worn monitor is configured to be worn on the patient's chest.

7. The system of claim 6, wherein the body-worn monitor is configured to attach to the patient's chest with a collection of electrode patches.

8. The system of claim 7, wherein the collection of electrode patches consists of two separate electrode patches.

9. The system of claim 8, wherein each electrode patch comprises two electrodes.

10. The system of claim 9, wherein each electrode patch comprises two electrodes connected to a common adhesive backing.

11. The system of claim 6, wherein the body-worn monitor comprises two separate modules, each comprising an electronics circuit and configured to be worn in the patient's chest.

12. The system of claim 11, wherein the body-worn monitor comprises a first module that houses an ECG circuit for measuring analog ECG waveforms used to calculate HR from the patient, and a second module that houses a TBI circuit for measuring analog TBI waveforms used to calculate CO and SV from the patient.

13. The system of claim 12, wherein the first and second modules are connected to each other with a cable.

14. The system of claim 12, wherein the body-worn monitor comprises a single analog-to-digital converter that converts the analog ECG waveforms into digital ECG waveforms, and the analog TBI waveforms into digital TBI waveforms.

15. The system of claim 14, wherein the body-worn monitor comprises a microprocessor that processes the digital ECG waveforms to determine an HR value.

16. The system of claim 14, wherein the body-worn monitor comprises a microprocessor that processes the digital TBI waveforms to determine an SV value.

17. The system of claim 14, wherein the body-worn monitor comprises a single microprocessor that processes the digital ECG waveforms to determine an HR value, and the digital TBI waveforms to determine an SV value.

18. The system of claim 1, wherein the body-worn monitor comprises a wireless system configured to transmit information to the data-processing software system.

19. The system of claim 18, wherein the body-worn monitor comprises a wireless system configured to transmit information to a mobile telephone, which includes a software application configured to transmit information to the computer system.
